(19) United States
(12) Patent Application Publication
(10) Pub. No.: US 2005/0014754 A1
(43) Pub. Date: Jan. 20, 2005

(54) Fused pyrrole compounds

(76) Inventors: Mitsunori Ono, Lexington, MA (US); Lijun Sun, Harvard, MA (US); ZhiQiang Xia, Acton, MA (US); Elena Kostik, Arlington, MA (US); Keizo Koya, Chestnut Hill, MA (US); Masazumi Nagai, Lexington, MA (US); Yaming Wu, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/799,389

(22) Filed: Mar. 12, 2004

Related U.S. Application Data
(60) Provisional application No. 60/454,963, filed on Mar. 13, 2003.

(51) Int. Cl. C07D 455/02; C07D 487/04; A61K 31/53; A61K 31/519

(52) U.S. Cl. 514/243; 544/183; 544/281; 546/138; 514/259.1; 514/306

(57) ABSTRACT

This invention relates to fused pyrrole compounds of Formula (I): 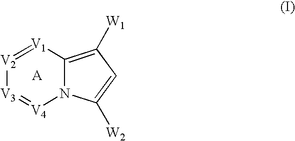
and pharmaceutically acceptable salts and prodrugs thereof, wherein V₁, V₂, V₃, V₄, R₁, R₂, R₃, W₁ and W₂ are as defined herein. The compounds of this invention may be used to prevent or treat cancer, inflammatory disorders, autoimmune diseasesand other conditions involving PDE4 or elevated levels of cytokines. The invention also provides methods of using the compounds to treat or prevent such disorders and pharmaceutical compositions containing the compounds.

### 1. CROSS REFERENCE TO RELATED APPLICATION

Pursuant to 35 USC § 119(e), this application claims priority to U.S. Provisional Application Ser. No. 60/454,963, filed Mar. 13, 2003, the contents of which are incorporated herein by reference.

### 2. FIELD OF THE INVENTION

This invention relates to novel, biologically active chemical compounds, namely fused pyrroles.

### 3. BACKGROUND OF THE INVENTION

Significant resources have been devoted to research for effective agents against cancer, inflammatory disorders and autoimmune diseases. Despite considerable advances, however, treatments for these conditions are inadequate for a number of reasons.

For example, there are still cancers which simply do not respond or respond poorly to treatments are currently available. Patients with treatable cancers must often undergo chemotherapy with drugs that cause severe side effects. Few of these drugs can be used orally. Perhaps the most serious problem associated with cancer chemotherapy is the development of multi-drug resistance by many tumors. For example, many tumors which initially respond positively to an anti-cancer therapy by decreasing in size or even going into remission often develop resistance to the drug. Tumors that have developed resistance to more than one drug are said to be a “multi-drug resistant”. There is little that can be done to halt or retard further progression of the disease, once a patient's cancer has become multi-drug resistant.

Recent studies have revealed that inhibition of the production or action of tumor necrosis factor alpha (TNFα) has therapeutic effects against inflammatory disorders and autoimmune diseases such as multiple sclerosis, pulmonary fibrosis, atherosclerosis, and Crohn's disease. See Newton et al., J. Med. Chem. (1999) 42(13): 2295-2314. TNFα also plays an important role as a proinflammatory mediator in the development and progression of heart failure. See Mann, D. L., Circ. Res. (2002) 91:988-998. The activity of TNFα can be inhibited by antibodies. However, this immunotherapy can be expensive and inconvenient to treat chronic diseases because the antibodies are administered intravenously once or twice a month in a hospital. Also, antibodies, like most other proteins, tend to be unstable after administration.

Preclinical and clinical studies on phosphodiesterase 4 (PDE4) inhibitors have demonstrated that these agents may find utility in a wide range of inflammatory disorders, including asthma, chronic obstructive pulmonary disease, atopic dermatitis, rheumatoid arthritis, multiple sclerosis, and various neurological disorders. See Doherty, A. M., Current Opinion in Chemical Biology (1999) 3:466-473. No PDE4 inhibitors have been used as drugs to treat inflammatory diseases.

There is therefore still a need for new drugs which overcome one or more of the aforementioned shortcomings of drugs currently used in the treatment of cancer, inflammatory disorders and autoimmune diseases. Desirable properties of new drugs therefore include efficacy against diseases or disorders that are currently untreatable or poorly treatable (e.g., efficacy against multi-drug resistant cancers), oral bioavailability and/or reduced side effects.

### 4. SUMMARY OF THE INVENTION

This invention is based on the discovery that certain fused pyrrole compounds are effective in preventing and treating cancer, inflammatory disorders, autoimmune diseases and other conditions involving PDE4 or elevated levels of cytokines.

In one embodiment, this invention features fused pyrrole compounds of Formula (I): 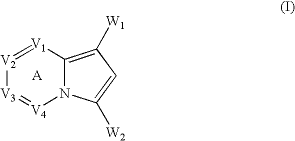
wherein:
one of W₁ and W₂ is 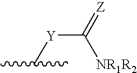
   and the other is 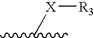
V₁, V₂, V₃ and V₄ are independently CR₆ or N; or alternatively, V₁ and V₂ taken together or V₃ and V₄ taken together may be replaced with S, O, or NR₇ to form a fused 5-membered heterocyclic ring, and wherein two adjacent positions on Ring A may optionally be joined to create a fused aryl group, provided that when W₁ is 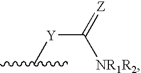
   V₁, V₂, V₃ and V₄ may not all be CR₆;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
Y is —C(R₄R₅)—, —N(R₄)—, —O—, —S—, —S(O)—, —S(O)₂—, —C(═O)—, —C(═S)—, —C(═O)—N(R₄)—, —C(═N—OR₈)—, —C(═N—R₈)—, or —N(R₄)—C(═O)—;
Z is ═O, ═S, ═N—OR₈ or ═NR₈;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, or alternatively, NR₁R₂, taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group;
each R₄ and R₅ is independently —H or a substituted or unsubstituted aliphatic group;
each R₆ is independently —H or a Ring A substituent;
each R₇ is independently —H or a heteroaryl ring nitrogen substituent and each R₈ is independently —H, an unsubstituted aliphatic group, a substituted aliphatic group, an unsubstituted non-aromatic heterocylic group, a substituted non-aromatic heterocylic group, an unsubstituted aryl group, or a substituted aryl group;
and pharmaceutically acceptable salts and prodrugs thereof.

One embodiment of the present invention relates to pharmaceutical compositions comprising a pharmaceutically acceptable carrier and a compound represented by Formula (I). These pharmaceutical compositions may be used in prophylasis or therapy, for example, to prevent or treat cancer, an inflammatory disorder, an autoimmune disease or other conditions involving PDE4 or elevated levels of cytokines.

Another embodiment of the present invention relates to the use of a compound represented by Formula (I) for the manufacture of a medicament for the prevention or treatment of cancer, an inflammatory disorder, an autoimmune disease or other conditions involving PDE4 or elevated levels of cytokines. The medicament comprises an effective amount of the compound.

Another embodiment of this invention relates to a method of treating a subject with cancer, an inflammatory disorder, an autoimmune disease or other conditions involving PDE4 or elevated levels of cytokines. The method comprises administering to the subject an effective amount of a compound represented by Formula (I) or a pharmaceutical composition comprising a compound represented by Formula (I).

Another embodiment of this invention relates to a method of preventing cancer, an inflammatory disorder, an autoimmune disease and other conditions involving PDE4 or elevated levels of cytokines in a subject susceptible to such disorder, disease or condition. The method comprises administering to the subject an effective amount of a compound represented by Formula (I) or a pharmaceutical composition comprising a compound represented by Formula (I).

Another embodiment of this invention relates to a method of inhibiting TNFα or PDE4 in a cell by contacting the cell with an effective amount of a compound represented by Formula (I) or a pharmaceutical composition comprising a compound represented by Formula (I).

Another embodiment of this invention relates to a method for reducing TNFα levels in a subject comprising administering to the subject an effective amount of a compound represented by Formula (I) or a pharmaceutical composition comprising a compound represented by Formula (I).

Another embodiment of this invention relates to a method for suppressing inflammatory cell activation comprising the step of contacting the cell with an effective amount of a compound represented by Formula (I) or a pharmaceutical composition comprising a compound represented by Formula (I).

Another embodiment is a method of preparing an intermediate in the synthesis of certain compounds represented by Formula (I). The intermediate is represented by Formula (I_{INT-A}):

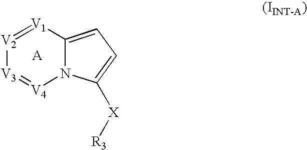

The method comprises the step of reacting a Cu^{I} salt with a precursor compound represented by Formula (I_{INT-B}): 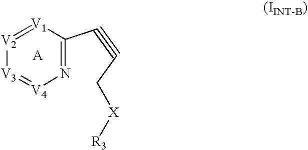
wherein in Formulas (I_{INT-A}) and (I_{INT-B}), Ring A, V₁, V₂, V₃, V₄, X, and R₃ are as described for Formula (I). In this method, R₃ is preferably not a substituted or unsubstituted alkyl group and more preferably, R₃ is a substituted or unsubstituted aryl group.

The fused pyrroles of this invention may have several benefits when used to treat or prevent cancer, inflammatory disorders, autoimmune diseases or other conditions involving elevated cytokine levels. For example, in the case of cancer, the compounds may be cytotoxic to many multi-drug resistant cell lines and therefore can be used when other traditional cancer chemotherapies have failed. In addition, the compounds may exhibit minimal side effects and may be active when administered orally. In the case of inflammatory disorders, autoimmune diseases and other conditions involving elevated cytokine levels, the fused pyrroles of this invention may provide enhanced efficacy, fewer side effects, and/or improved dosing options.

The details of various embodiments of the invention are set forth in the description below. Other features, objects, and advantages of the invention will be apparent from the following description and from the claims.

### 5. DETAILED DESCRIPTION OF THE INVENTION

5.1. Definitions

Unless otherwise specified, the below terms (and terms analogous or similar thereto) as used herein are defined as follows:

The term “aryl group” refers to carbocyclic or heterocyclic aromatic groups (typically a 5-8 membered monocyclic aromatic ring or a polycyclic aromatic ring or ring system having 5-8 ring members in each ring thereof), such as phenyl, naphthyl, and anthracyl, and heteroaryl groups such as imidazolyl, isoimidazolyl, thienyl, furanyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, and tetrazolyl. Aryl groups also include fused polycyclic aromatic ring systems in which a carbocyclic aromatic ring or heteroaryl ring is fused to one or more other carbocyclic aromatic or heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl. Nitrogen-containing aryl groups (such as pyridyl) expressly include their N-oxide forms.

An “aliphatic group” is a straight chained, branched or cyclic non-aromatic hydrocarbon which is completely saturated or which contains one or more units of unsaturation. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. A C₁-C₄ straight chained or branched alkyl group or a C₃-C₈ cyclic alkyl group is also referred to as a “lower alkyl” group. For the purpose of this invention, “aliphatic group”, “alkyl” and other terms that incorporate those terms as a prefix or suffix (e.g., alkoxy and aminoalkyl) also includes those moieties where one or more carbons in the group are substituted with oxygen (O), sulfur (S), or nitrogen (N). Further, those groups may optionally be substituted with one or more conventionally used alkyl substituents, such as amino, alkylamino, alkoxy, alkylthio, oxo, halo, acyl, nitro, hydroxyl, cyano, aryl, alkylaryl, aryloxy, arylthio, arylamino, carbocyclyl, carbocyclyloxy, carbocyclylthio, carbocyclylamino, heterocyclyl, heterocyclyloxy, heterocyclylamino, heterocyclylthio, and the like.

An “alkylene group” is represented by —(CH₂)ₙ—, wherein n is an integer from 1-10, preferably 1-4 and substituted and branched variants thereof. “Non-aromatic heterocyclic” rings or groups are non-aromatic carbocyclic rings or ring systems which include one or more heteroatoms such as nitrogen, oxygen or sulfur in the ring. Typically, the ring may be five, six, seven or eight-membered or if fused, each ring of the system may have five, six, seven or eight members. Examples include oxazolinyl, thiazolinyl, oxazolidinyl, thiazolidinyl, tetrahydrofuranyl, tetrahyrothiophenyl, morpholino, thiomorpholino, pyrrolidinyl, piperazinyl, piperidinyl, and thiazolidinyl. The terms “heterocyclyl”, “heterocyclic” and the like include both non-aromatic and aromatic heterocycles.

Suitable substituents for Ring A, an aliphatic group, aryl group, or non-aromatic heterocyclic group are those which do not substantially interfere with the prophylactic or therapeutic activity of the disclosed compounds. Examples of suitable substituents include, but are not limited to, —OH, halogen (—Br, —Cl, —I and —F), —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NRCCONR^{a}H, —NRCCON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}C(═NH)—N(R^{a}R^{b}), —NR^{d}—C(═NR^{c})—NH₂, —NR^{d}—C(═NR^{c})—NHR^{a}, —NR^{d}C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CRC═CR^{a}R^{b}, —CR^{c}═CHR^{a}, —CRC═CR^{a}R^{b}, —CCR^{a}, —SH, SR^{a}, —SOₖR^{a} (k is 0, 1 or 2) and —NH—C(═NH)—NH₂. R^{a}—R^{d} are each independently an aliphatic, substituted aliphatic, benzyl, substituted benzyl, aryl or substituted aryl group, preferably an alkyl, benzylic or aryl group. In addition, —NR^{a}R^{d}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent, which may be the same or different.

Suitable substituents for heteroaryl ring nitrogen atoms having three covalent bonds to other heteroaryl ring atoms include —OH and -alkoxy (preferably C₁-C₄). Substituted heteroaryl ring nitrogen atoms that have three covalent bonds to other heteroaryl ring atoms are positively charged, which is balanced by counteranions such as chloride, bromide, formate, acetate and the like. Examples of other suitable counteranions are provided in the section below directed to suitable pharmacologically acceptable salts.

Suitable substituents for heteroaryl ring nitrogen atoms having two covalent bonds to other heteroaryl ring atoms include alkyl, substituted alkyl (including haloalkyl), phenyl, substituted phenyl, —S(O)₂-(alkyl), —S(O)₂—NH(alkyl) and —S(O)₂—NH(alkyl)₂.

Preferred substituents for carbon atoms on Ring A include aryl (e.g., optionally substituted phenyl), halo (e.g., —F, —Cl, and —Br), —C₁-C₄ alkyl, —C₁-C₄ alkoxy, —C₁-C₄ alkoxycarbonyl, —C₁-C₄ haloalkyl, —C₁-C₄ haloalkoxy, —C₁-C₄ haloalkoxycarbonyl, —C₁-C₄ acyl, amido, substituted amido, NO₂, —CN, —OH, —NH₂ and substituted amino. Preferred substituents for nitrogen atoms on Ring A include aryl (e.g., optionally substituted phenyl), —C₁-C₄ alkyl, —C₁-C₄ alkoxycarbonyl, —C₁-C₄ haloalkyl, —C₁-C₄ haloalkoxycarbonyl, and —C₁-C₄ acyl and substituted amino. Ring A can have zero, one or more substituents. For substituted amido and substituted amino, the preferred substituent is lower alkyl.

Preferred substitutents for Rings D-T include C₁-C₄ alkyl, C₁-C₄ hydroxyalkyl, N-morpholino, pyrimidyl, C₁-C₄ alkyl substituted with pyrimidyl, —N(C₁-C₄ alkyl)₂, —C(O)NH₂, —C(O)NH(C₁-C₄ alkyl), C(O)N(C₁-C₄ alkyl)₂, —NHC(O)(C₁-C₄ alkyl), —NO₂, C₁-C₄ alkoxy, —C(O)O—CH₂CH₂—N(C₁-C₄ alkyl)₂, 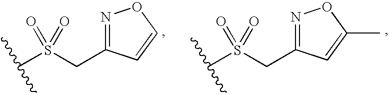
—NH-(phenyl), —NH₂, —CH₂NH—C(O)—O—(C₁-C₄ alkyl), —CH₂NH₂, —Cl, —F, —C(O)—O—(C₁-C₄ alkyl), —C(O)—N—(C₁-C₄ alkyl), C₃-C₇ cycloalkyl, phenyl, —C(O)—N-morpholino, —S—(C₁-C₄ alkyl), —CN, furyl, —S(O)₂—(C₁-C₄ alkyl), —S(O)₂—NH₂, —S(O)₂—NH(C₁-C₄ alkyl) or —S(O)₂—N(C₁-C₄ alkyl)₂.

Also included in the present invention are pharmaceutically acceptable salts of the compounds described herein. Compounds disclosed herein which possess a sufficiently acidic, a sufficiently basic, or both functional groups, and accordingly can react with any of a number of organic or inorganic bases, and inorganic and organic acids, to form a salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Prodrugs of the compounds of this invention are also contemplated herein. The term “prodrug” means a derivative of a compound that can hydrolyze, oxidize, or otherwise react under biological conditions (in vitro or in vivo) to provide a compound of this invention. Prodrugs may only become active upon such reaction under biological conditions, but they may have activity in their unreacted forms. Examples of prodrugs contemplated in this invention include, but are not limited to, analogs or derivatives of compounds of Formula (I) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of Formula (I) that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, a-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, amino acids, hydroxyalkylamines, heterocyclic and heteroaromatic amines, and polyether amines.

Certain compounds of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures.

As used herein, a racemic mixture means about 50% of one enantiomer and about 50% of is corresponding enantiomer relative to all chiral centers in the molecule. The invention encompasses all enantiomerically-pure, enantiomerically-enriched, diastereomerically pure, diastereomerically enriched, and racemic mixtures of the compounds of Formula (I).

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

Where a particular substituent occurs multiple times in a given structure, the identity of the substitutent is independent in each case and may be the same as or different from other occurrences of that substituent in the structure. Furthermore, individual substituents in the exemplary compounds shown below are preferred in combination with other substituents in the compounds of this invention, even if such substituents are not expressly noted as being preferred or not expressly shown in combination with other substituents.

The term “cytokine,” as used herein, means any secreted polypeptide that affects the functions of other cells, and that modulates interactions between cells in the immune or inflammatory response. Cytokines include, but are not limited to monokines, lymphokines, and chemokines regardless of which cells produce them. For instance, a monokine is generally referred to as being produced and secreted by a monocyte, however, many other cells produce monokines, such as natural killer cells, fibroblasts, basophils, neutrophils, endothelial cells, brain astrocytes, bone marrow stromal cells, epidermal keratinocytes, and B-lymphocytes. Lymphokines are generally referred to as being produced by lymphocyte cells. Examples of cytokines include, but are not limited to, interleukin-1 (IL-1), interleukin-6 (IL-6), Tumor Necrosis Factor alpha (TNFa), and Tumor Necrosis Factor beta (TNFβ).

The present invention further provides a method of reducing TNFα levels in a subject, comprising the step of administering an effective amount of a compound of Formula (I) to the subject. The term “reducing TNFα levels,” as used herein, means either:
a) decreasing excessive in vivo TNFa levels in a mammal to normal levels or below normal levels by inhibition of the in vivo release of TNFα by all cells, including but not limited to monocytes or macrophages; or
b) inducing a down-regulation, at the translational or transcription level, of excessive in vivo TNFα levels in a mammal to normal levels or below normal levels; or
c) inducing a down-regulation, by inhibition of the direct synthesis of TNFα as a postranslational event.

Moreover, the compounds of the present invention are useful in suppressing inflammatory cell activation. The term “inflammatory cell activation,” as used herein, means the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes, polymorphonuclear leukocytes, mast cells, basophils, eosinophils, dendritic cells, and endothelial cells). It will be appreciated by persons skilled in the art that the activation of one or a combination of these phenotypes in these cells can contribute to the initiation, perpetuation, or exacerbation of an inflammatory condition.

Without wishing to be bound by theory, the compounds of this invention inhibit TNFα and/or PDE4. In this context “inhibit” refers to interfering with the production or activity of TNFα or PDE4 in a direct or an indirect fashion. For example, the compounds of this invention may block production of TNFα by interfering at the transcriptional, translational or post-translational level or blocking the activity of the PDE4 enzyme. In some cases, compounds of this invention will inhibit TNFa but not PDE4. The ability of a compound of this invention to inhibit TNFa and/or PDE4 may be readily evaluated using the techniques described herein and other techniques known to those of skill in the art.

The compounds of this invention can be used to treat subjects with cancer, including multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, typically five or more.

As used herein, the term “cancer” means a disease, condition or disorder characterized by a proliferation of cells with loss of normal controls resulting in unregulated growth, lack of differentiation, local tissue invasion, and metastasis. Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. Pre-malignant abnormal cell growth is exemplified by hyperplasia, metaplasia, or most particularly, dysplasia (for review of such abnormal growth conditions, see Robbins and Angell (1976) Basic Pathology, 2d Ed., W. B. Saunders Co., Philadelphia, 68-79.) The compounds of this invention may be used to prevent or treat cancer in each of these cases and the term “cancer” as used herein encompasses all such abnormal growth conditions whether they are considered cancerous or pre-cancerous.

Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. The neoplastic lesion may evolve clonally and develop an increasing capacity for invasion, growth, metastasis, and heterogeneity, especially under conditions in which the neoplastic cells escape the host's immune surveillance (Roitt, I., Brostoff, J and Kale, D. (1993) Immunology, 3rd ed., Mosby, St. Louis, 17.1-17.12).

Cancers that can be treated or prevented by the compounds and methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In the case of cancer, the term “treating” includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

As used herein, the term “asthma” means a pulmonary disease, disorder or condition characterized by reversible airway obstruction, airway inflammation, and increased airway responsiveness to a variety of stimuli.

The compounds of this invention can be used to treat subjects with autoimmune diseases. As used herein, the term “autoimmune disease” means a disease, disorder or condition caused by the immune system of an animal mistakenly attacking itself, thereby targeting the cells, tissues, and/or organs of the animal's own body. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus. Specific autoimmune diseases that may be ameliorated using the compounds and methods of this invention include without limitation, autoimmune diseases of the nervous system (e.g., multiple sclerosis, myasthenia gravis, autoimmune neuropathies such as Guillain-Barré, and autoimmune uveitis), autoimmune diseases of the blood (e.g., autoimmune hemolytic anemia, pernicious anemia, and autoimmune thrombocytopenia), autoimmune diseases of the blood vessels (e.g., temporal arteritis, anti-phospholipid syndrome, vasculitides such as Wegener's granulomatosis, and Behcet's disease), autoimmune diseases of the skin (e.g., psoriasis, dermatitis herpetiformis, pemphigus vulgaris, and vitiligo), autoimmune diseases of the gastrointestinal system (e.g., Crohn's disease, ulcerative colitis, primary biliary cirrhosis, and autoimmune hepatitis), autoimmune diseases of the endocrine glands (e.g., Type 1 or immune-mediated diabetes mellitus, Grave's disease. Hashimoto's thyroiditis, autoimmune oophoritis and orchitis, and autoimmune disease of the adrenal gland); and autoimmune diseases of multiple organs (including connective tissue and musculoskeletal system diseases) (e.g., rheumatoid arthritis, systemic lupus erythematosus, scleroderma, polymyositis, dermatomyositis, spondyloarthropathies such as ankylosing spondylitis, and Sjogren's syndrome). In addition, other immune system mediated diseases, such as graft-versus-host disease and allergic disorders, are also included in the definition of autoimmune diseases herein. Because a number of autoimmune disorders are caused by inflammation, there is some overlap between disorders that are considered autoimmune diseases and inflammatory disorders. For the purpose of this invention, in the case of such an overlapping disorder, it may be considered either an autoimmune disease or an inflammatory disorder. “Treatment of an autoimmune disease” herein refers to administering a composition of the invention to a subject, who has an autoimmune disease, a symptom of such a disease or a predisposition towards such a disease, with the purpose to cure, relieve, alter, affect, or prevent the autoimmune disease, the symptom of it, or the predisposition towards it.

As used herein, the term “allergic disorder” means a disease, condition or disorder associated with an allergic response against normally innocuous substances. These substances may be found in the environment (such as indoor air pollutants and aeroallergens) or they may be non-environmental (such as those causing dermatological or food allergies). Allergens can enter the body through a number of routes, including by inhalation, ingestion, contact with the skin or injection (including by insect sting). Many allergic disorders are linked to atopy, a predisposition to generate the allergic antibody IgE. Because IgE is able to sensitize mast cells anywhere in the body, atopic individuals often express disease in more than one organ. For the purpose of this invention, allergic disorders include any hypersensitivity that occurs upon re-exposure to the sensitizing allergen, which in turn causes the release of inflammatory mediators. Allergic disorders include without limitation, allergic rhinitis (e.g., hay fever), sinusitis, rhinosinusitis, chronic or recurrent otitis media, drug reactions, insect sting reactions, latex reactions, conjunctivitis, urticaria, anaphylaxis and anaphylactoid reactions, atopic dermatitis, asthma and food allergies.

The compounds of this invention can be used to prevent or to treat subjects with inflammatory disorders. As used herein, an “inflammatory disorders” means a disease, disorder or condition characterized by inflammation of the body tissue. These include local inflammatory responses and systemic inflammation. Examples of such inflammatory disorders include: transplant rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis); as well as various other diseases with significant inflammatory components, including preeclampsia; chronic liver failure, brain and spinal cord trauma, cancer). There may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy. “Treatment of an inflammatory disorder” herein refers to administering a composition of the invention to a subject, who has an inflammatory disorder, a symptom of such a disorder or a predisposition towards such a disorder, with the purpose to cure, relieve, alter, affect, or prevent the inflammatory disorder, the symptom of it, or the predisposition towards it.

An “effective amount” is the quantity of compound in which a beneficial outcome is achieved when the compound is administered to a subject or alternatively, the quantity of compound that possess a desired activity in-vivo or in-vitro. In the case of cancer, a beneficial clinical outcome includes a reduction in tumor mass, a reduction in the rate of tumor growth, a reduction in metastasis, a reduction in the severity of the symptoms associated with the cancer and/or an increase in the longevity and/or quality of life of the subject compared with the absence of the treatment. In the case of inflammatory disorders and autoimmune diseases, a beneficial clinical outcome includes reduction in the extent or severity of the symptoms associated with the disease or disorder and/or an increase in the longevity and/or quality of life of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of cancer, inflammatory disorder or autoimmune disease. The skilled artisan will be able to determine appropriate dosages depending on these and other factors. Effective amounts of the disclosed compounds typically range between about 1 mg/mm² per day and about 10 grams/mm² per day, and preferably between 10 mg/mm² per day and about 5 grams/mm².

The disclosed compounds are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), or rectally, depending on the type of cancer to be treated. Oral or parenteral administration are preferred modes of administration.

The disclosed compounds can be administered to the subject in conjunction with an acceptable pharmaceutical carrier, adjuvant, diluent, excipient or solvent as part of a pharmaceutical composition. For convenience, the term “carrier” will encompass all such carriers, adjuvants, diluents, excipients, solvents or other inactive additives. Formulation of the compound to be administered will vary according to the route of administration selected (e.g., solution, emulsion, capsule) and the disease, condition or disorder targeted. Suitable pharmaceutical carriers may contain inert ingredients which do not substantially interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

Optionally, the disclosed compounds can be co-administered with other anti-cancer agents such as Taxol, Vincristine, Adriamycin, Etoposide, Doxorubicin. Dactinomycin, Mitomycin C, Bleomycin, Vinblastine, Cisplatin and the like. Preferably, the disclosed compounds are co-administered before the cancer develops multi-drug resistance or as the cancer is developing multi-drug resistance but before the cancer becomes completely resistant to the anticancer drugs being used. The method can also be carried in combination with other cancer treatments such as surgery, radiation, and the like.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The compounds of this invention may be used to treat or prevent “other conditions involving PDE4 or elevated levels of cytokines”. This term includes, but is not limited to, any disease, condition or disorder which is characterized, mediated or exacerbated by overproduction or activity of TNFα. In addition, this term includes, without limitation, any disease, condition or disorder which is characterized, mediated or exacerbated by overproduction or activity of PDE4 (whether or not it results in elevated level of cytokines). Such conditions include many types of inflammatory disorders, including inflammatory bowel disease (e.g., Crohn's disease), asthma, sepsis, stroke, heart failure, chronic obstructive pulmonary disease, allergic rhinitis, and autoimmune diseases (e.g., arthritis, multiple sclerosis, atherosclerosis, and psoriasis), but will also include other categories of diseases (including, without limitation, cardiomyopathies, such as congestive heart failure, pyrexia, cachexia, cachexia secondary to infection or malignancy, cachexia secondary to acquired immune deficiency syndrome (AIDS), ARC (AIDS-related complex), cerebral malaria, osteoporosis and bone resorption diseases, and fever and myalgias due to infection. In addition, the compounds of the present invention are useful in the treatment of diabetes insipidus and central nervous system disorders, such as depression and multi-infarct dementia).

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a reaction that is “substantially complete” means that the reaction contains more than about 80% by weight of the desired product, more preferably more than about 90% by weight of the desired product, even more preferably more than about 95% by weight of the desired product, and most preferably more than about 97% by weight of the desired product.

As used herein, a racemic mixture means about 50% of one enantiomer and about 50% of is corresponding enantiomer relative to all chiral centers in the molecule. The invention encompasses all enantiomerically-pure, enantiomerically-enriched, diastereomerically pure, diastereomerically enriched, and racemic mixtures of the compounds of Formula (I).

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds of the invention are defined herein by their chemical structures and/or chemical names. Where a compound is referred to by both a chemical structure and a chemical name, and the chemical structure and chemical name conflict, the chemical structure is determinative of the compound's identity.

When administered to a patient, e.g., to a non-human animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least about 90%, preferably at least about 95% or more preferably, at least about 98%, of a compound of this invention by weight of the isolate.

As used herein, a composition that is “substantially free” of a compound means that the composition contains less than about 20% by weight, more preferably less than about 10% by weight, even more preferably less than about 5% by weight, and most preferably less than about 3% by weight of the compound.

Choices and combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject). Typically, such compounds are stable at a temperature of 40° C. or less, in the absence of excessive moisture, for at least one week. Such choices and combinations will be apparent to those of ordinary skill in the art and may be determined without undue experimentation.

5.2. Chemical Structures

This invention features fused pyrrole compounds of Formula (I): 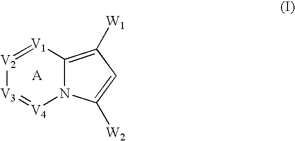
wherein:
one of W₁ and W₂ is 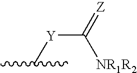
   and the other is 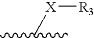
V₁, V₂, V₃ and V₄ are independently CR₆ or N; or alternatively, V₁ and V₂ taken together or V₃ and V₄ taken together may be replaced with S, O, or NR₇ to form a fused 5-membered heterocyclic ring, and wherein two adjacent positions on Ring A may optionally be joined to create a fused aryl group, provided that when W₁ is 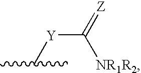
   V₁, V₂, V₃ and V₄ may not all be CR₆;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
Y is —C(R₄R₅)—, —N(R₄)—, —O—, —S—, —S(O)—, —S(O)₂—, —C(═O)—, —C(═S)—, —C(═O)—N(R₄)—, —C(═N—OR₈)—, —C(═N—R₈)—, or —N(R₄)—C(═O)—;
Z is ═O, ═S, ═N—OR₈ or ═NR₈;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, or alternatively, NR₁R₂, taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group;
each R₄ and R₅ is independently —H or a substituted or unsubstituted aliphatic group;
each R₆ is independently —H or a Ring A substituent;
each R₇ is independently —H or a heteroaryl ring nitrogen substituent and
each R₈ is independently —H, an unsubstituted aliphatic group, a substituted aliphatic group, an unsubstituted non-aromatic heterocylic group, a substituted non-aromatic heterocylic group, an unsubstituted aryl group, or a substituted aryl group;
and pharmaceutically acceptable salts and prodrugs thereof.

One specific embodiment provides the compound of Formula (I) wherein each R₆ is independently selected from H, halo, —C₁-C₄ alkyl, —C₁-C₄ alkoxy, —C₁-C₄ haloalkyl, C₁-C₄ haloalkoxy, —C₁-C₄ acyl, amido, substituted amido, —NO₂, —CN, —OH, —NH₂ and substituted amino; Y is —C(R₄R₅)— or C═O; Z is ═O; R₁ is —H; R₂ is a substituted or unsubstituted alkyl group or a substituted or unsubstituted aryl group; R₃ is a substituted or unsubstituted aryl group; each R₈ is independently —H or a substituted or unsubstituted aliphatic group, and X is —C(R₄R₅)—, —N(R₄)—, —C(═O)— or —O—. In a subset of these compounds, R₄ and R₅ are both H, Y is C═O; and R₄ and R₅ are both H. In another specific embodiment, R₂ is an unsubstituted aryl group or an aryl group substituted with lower alkyl, amido, cyano, or halo; R₃ is a substituted or unsubstituted phenyl, a substituted or unsubstituted pyridyl or a substituted or unsubstituted thienyl; each R₈ is independently —H or a substituted or unsubstituted lower alkyl and X is —CH₂—, —CH(lower alkyl)-, —NH—, —N(lower alkyl)-, —C(═O)— or —O—.

Examples of specific structures of Formula (I) include structures of Formulas (Ia)-(Ig): 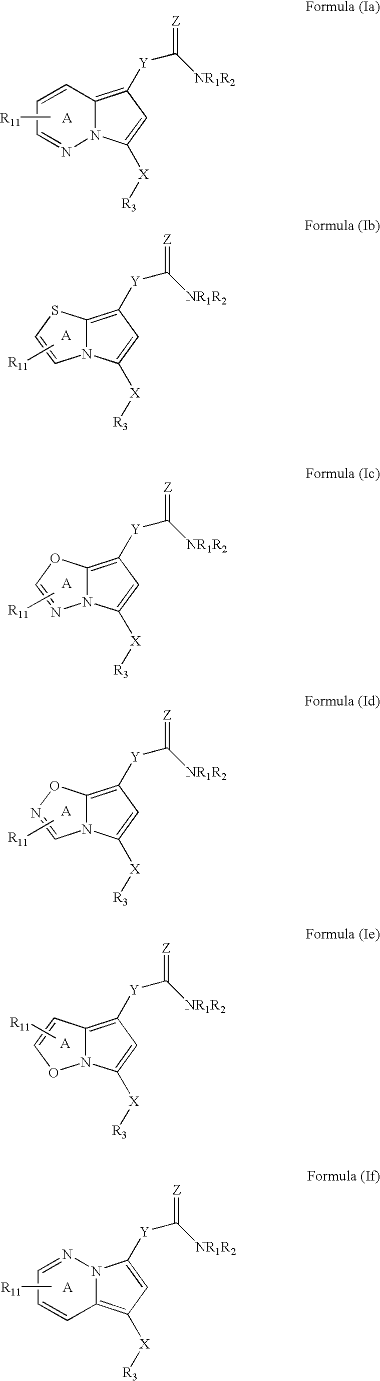 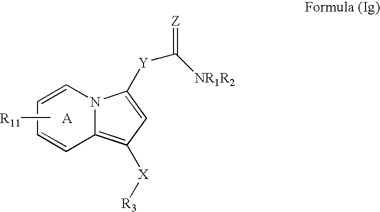
wherein Ring A, X, Y, Z, R₁, R₂, and R₃ are as described above for Formula (I), R₁₁ occurs at each unfixed position of Ring A and each R₁₁ is independently selected from Ring A substituents (preferably, selected from the group consisting of H, hydroxyl, cyano, nitro, halo, a substituted or unsubstituted amino group, a substituted or unsubstituted acyl group, a substituted or unsubstituted amido group, a substituted or unsubstituted alkyl group, a substituted or unsubstituted alkoxy group, or a substituted or unsubstituted aryl group (and more preferably, H, halo, —C₁-C₄ alkyl, —C₁-C₄ alkoxy, —C₁-C₄ haloalkyl, C₁-C₄ haloalkoxy, —C₁-C₄ acyl, amido, substituted amido, —NO₂, —CN, —OH, —NH₂ and substituted amino)).

In one embodiment, Y is —C(R₄R₅)— or C═O; Z is ═O; R₁ is —H; R₂ is a substituted or unsubstituted alkyl group or a substituted or unsubstituted aryl group; R₃ is a substituted or unsubstituted aryl group; X is —C(R₄R₅)—, —N(R₄)—, —C(═O)— or —O— (preferably, —C(R₄R₅)) and R₁₁ is as described above. In a subset of these compounds, and R₄ and R₅ are both H, Y is C═O. In another embodiment, R₃ is a substituted or unsubstituted phenyl, pyridyl or thienyl group.

As noted above, values for R₁—R₃ include substituted and unsubstituted aryl groups. For these substituents (and particularly for R₂), aryl groups include those represented by Formulas (II)-(XV):

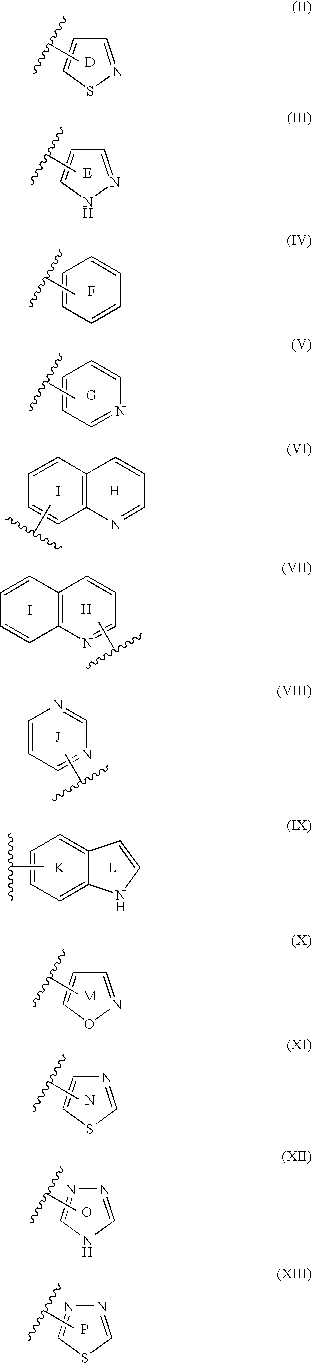

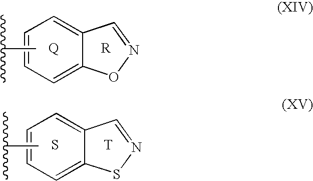

Rings D-T may be substituted or unsubstituted. Particular aryl groups for R₂ are represented by Formulas (XVI)-(XXI): 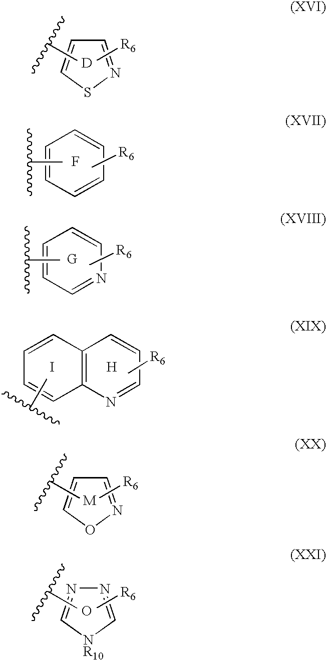
wherein R₆ occurs at each unfixed position in each Ring D, F, G, I, H, M, and O, each R₆ is independently selected from the group consisting of H, hydroxyl, cyano, nitro, halo, a substituted or unsubstituted alkyl group, a substituted or unsubstituted alkoxy group, or a substituted or unsubstituted aryl group, R₁₀ is —H or a substituted or unsubstituted alkyl group, and Rings D, F, G, I, H, M, and O are as described above.

Additional aryl groups for R₁, R₂, and R₃ (and in particular, R₂) are represented by Formulas (XXII)-(XXVII): 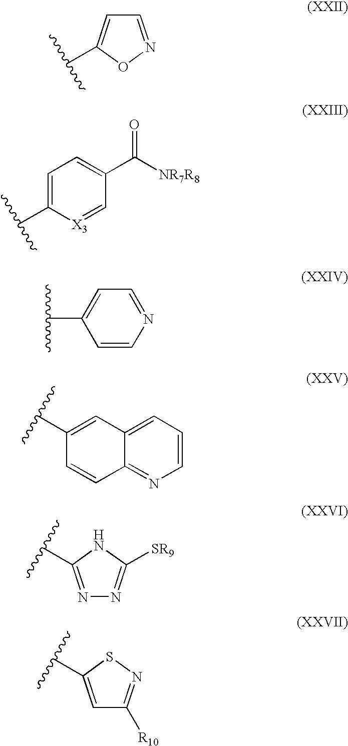
wherein X₃ is —CH— or —N—;
R₇ and R₈ are independently —H or a substituted or unsubstituted alkyl group or alternatively, —NR₇R₈, taken together, is a nitrogen-containing non-aromatic heterocyclic group;
R₉ is a substituted or unsubstituted alkyl group; and
R₁₀ is —H or a substituted or unsubstituted alkyl group.

5.3. Exemplary Compounds of the Invention

Exemplary compounds of the invention are depicted in Table 1 below.

TABLE 1

| No. | Compound | Name |
|---|---|---|
| I-1 | 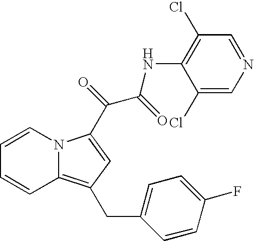 | N-(3,5-Dichloro-pyridin-4-yl)-2-[1-(4-fluoro- benzyl)-indolizin-3-yl]-2-oxo-acetamide |
| I-2 | 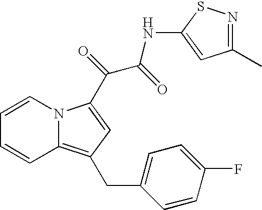 | 2-[1-(4-fluoro-benzyl)-indolizin-3-yl]-N-(3- methyl-isothiazol-5-yl)-2-oxo-acetamide |
| I-3 | 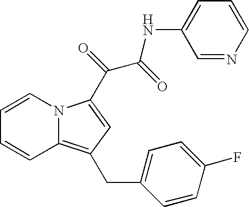 | 2-[1-(4-Fluoro-benzyl)-indolizin-3-yl]-2-oxo-N- pyridin-3-yl-acetamide |
| I-4 | 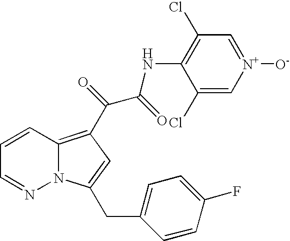 | N-(3,5-Dichloro-1-oxy-pyridin-4-yl)-2-[7-(4- fluoro-benzyl)-pyrrolo[1,2-b]pyridazin-5-yl]-2- oxo-acetamide |
| I-5 | 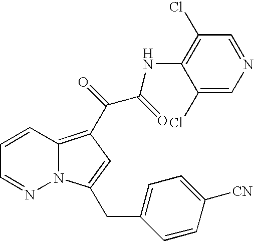 | 2-[7-(4-Cyano-benzyl)-pyrrolo[1,2-b]pyridazin- 5-yl]-N-(3,5-dichloro-pyridin-4-yl)-2-oxo- acetamide |
| I-6 | 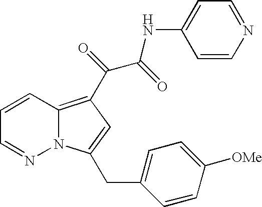 | 2-[7-(4-Methoxy-benzyl)-pyrrolo [1,2-b]pyridazin-5-yl]-2-oxo-N-pyridin-4-yl- acetamide |
| I-7 | 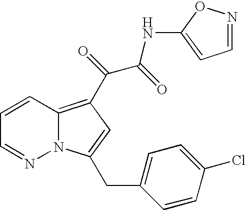 | 2-[7-(4-Chloro-benzyl)-pyrrolo [1,2-b]pyridazin-5-yl]-N-isoxazol-5-yl-2-oxo- acetamide |
| I-8 | 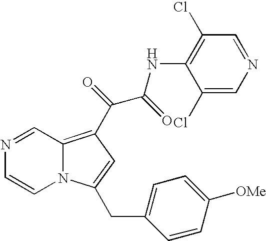 | N-(3,5-Dichloro-pyridin-4-yl)-2-[6-(4-methoxy- benzyl-pyrrolo[1,2-a]pyrazin-8-yl]-2-oxo- acetamide |
| I-9 | 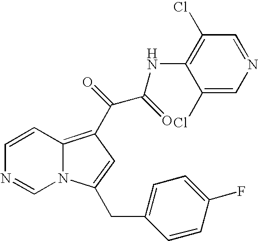 | N-(3,5-Dichloro-pyridin-4-yl)-2-[7-(4-fluoro- benzyl)-pyrrolo[1,2-c]pyrimidin-5-yl]-2-oxo- acetamide |
| I-10 | 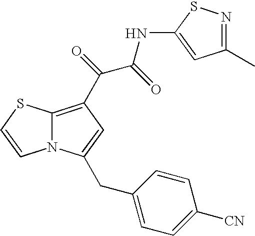 | 2-[5-(4-Cyano-benzyl)-pyrrolo[2,1-b]thiazol-7- yl]-N-(3-methyl-isothiazol-5-yl)-2-oxo- acetamide |
| I-11 | 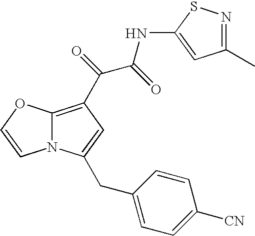 | 2-[5-(4-Cyano-benzyl)-pyrrolo[2,1-b]oxazol-7- yl]-N-(3-methyl-isothiazol-5-yl)-2-oxo- acetamide |
| I-12 | 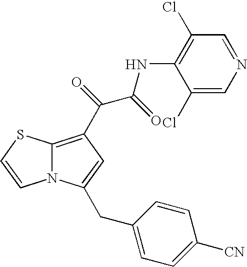 | 2-[5-(4-Cyano-benzyl)-pyrrolo[2,1-b]thiazol-7- yl]-N-(3,5-dichloro-pyridin-4-yl)-2-oxo- acetamide |
| I-13 | 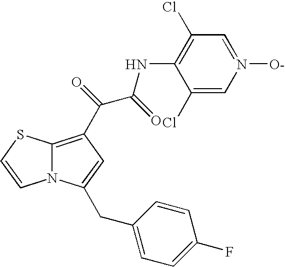 | N-(3,5-Dichloro-1-oxy-pyridin-4-yl)-2-[5-(4- fluoro-benzyl)-pyrrolo[2,1-b]thiazol-7-yl]-2- oxo-acetamide |
| I-14 | 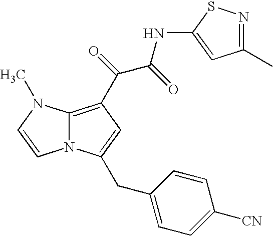 | 2-[5-(4-Cyano-benzyl)-1-methyl-1H-pyrrolo [1,2-a]imidazol-7-yl]-N-(3-methyl-isothiazol-5- yl)-2-oxo-acetamide |

5.4. Methods of Treatment and Prevention

In accordance with the invention, an effective amount of a compound of Formula (I) or a pharmaceutically acceptable salt or prodrug thereof, or a pharmaceutical composition comprising a compound of Formula (I) or a pharmaceutically acceptable salt or prodrug thereof, is administered to a subject in need of treatment or prevention of cancer, an inflammatory disorder, an autoimmune disease or other condition ameliorated by inhibition of TNFα and/or PDE4. The subject is preferably a mammal and more preferably, a human. Based on the disclosure herein, other conditions, diseases and disorders that would benefit from such uses will be apparent to those of skill in the art a subject, preferably a mammal and moe preferably, a human

In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder or symptoms thereof.

In certain embodiments, the compounds of the invention or the compositions of the invention are administered to a subject, preferably a mammal and more preferably, a human, as a prophylactic or preventative measure against particular conditions, diseases and disorders. As used herein, “prevention” or “preventing” refers to a reduction of the risk of acquiring a given condition, disease or disorder. In a preferred mode of the embodiment, the compositions of the present invention are administered as a preventative measure to a patient, preferably a human, having a genetic predisposition to any of the cancers, inflammatory disorders, autoimmune diseases or other conditions ameliorated by inhibition of TNFa and/or PDE4 described herein. In each of the therapeutic or prophylactic methods of the invention, a therapeutically or prophylactically effective amount of a compound of Formula (I) or a pharmaceutically acceptable salt or prodrug thereof is administered to a subject.

The compounds of Formula (I) and pharmaceutically acceptable salts and prodrugs thereof can be assayed in vitro or in vivo, for the desired therapeutic or prophylactic activity, prior to use in humans. For example, animal model systems can be used to demonstrate the safety and efficacy of compounds of this invention.

5.5. Pharmaceutical Compositions and Dosage Forms

Pharmaceutical compositions and dosage forms of the invention comprise one or more active ingredients in relative amounts and formulated in such a way that a given pharmaceutical composition or dosage form has the desired biological effect. Preferred pharmaceutical compositions and dosage forms comprise a compound of Formula (1), or a pharmaceutically acceptable salt or prodrug thereof, optionally in combination with one or more additional active agents. These compounds can be administered to the subject in conjunction with an acceptable pharmaceutical carrier, adjuvant, diluent, excipient, solvent or other additives as part of the pharmaceutical composition. For convenience, the term “carrier” will encompass all such carriers, adjuvants, diluents, excipients, solvents or other additives. Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990)18th ed., Mack Publishing, Easton Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines (e.g., N-desmethylvenlafaxine and N,N-didesmethylvenlafaxine) are particularly susceptible to such accelerated decomposition. Consequently, this invention encompasses pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmocopia (USP)SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen (1995) Drug Stability: Principles & Practice, 2d. Ed., Marcel Dekker, NY, N.Y., 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a compound of Formula (I), or a pharmaceutically acceptable salt or prodrug thereof in an amount of from about 1 mg to about 1000 mg, preferably in an amount of from about 50 mg to about 500 mg, and most preferably in an amount of from about 75 mg to about 350 mg. The typical total daily dosage of the compound of Formula (I), or a pharmaceutically acceptable salt or prodrug thereof can range from about 1 mg to about 5000 mg per day, preferably in an amount from about 50 mg to about 1500 mg per day, more preferably from about 75 mg to about 1000 mg per day. It is within the skill of the art to determine the appropriate dose and dosage form for a given subject.

5.5.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990)18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.5.2. Controlled Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

A particular extended release formulation of this invention comprises a therapeutically or prophylactically effective amount of a compound of Formula (I), or a pharmaceutically acceptable salt or prodrug thereof, in spheroids which further comprise microcrystalline cellulose and, optionally, hydroxypropylmethyl-cellulose coated with a mixture of ethyl cellulose and hydroxypropylmethylcellulose. Such extended release formulations can be prepared according to U.S. Pat. No. 6,274,171, the entirely of which is incorporated herein by reference.

A specific controlled-release formulation of this invention comprises from about 6% to about 40% a compound of Formula (I) by weight, about 50% to about 94% microcrystalline cellulose, NF, by weight, and optionally from about 0.25% to about 1% by weight of hydroxypropyl-methylcellulose, USP, wherein the spheroids are coated with a film coating composition comprised of ethyl cellulose and hydroxypropylmethylcellulose.

5.5.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

5.5.4. Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences (1980 & 1990) 16th and 18th eds., Mack Publishing, Easton Pa. and Introduction to Pharmaceutical Dosage Forms (1985) 4th ed., Lea & Febiger, Philadelphia. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990)16th and 18th eds., Mack Publishing, Easton Pa.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.5.5. Kits

This invention encompasses kits which, when used by the medical practitioner, can simplify the administration of appropriate amounts of active ingredients to a patient.

A typical kit of the invention comprises a unit dosage form of a compound of Formula (I), or a pharmaceutically acceptable prodrug or salt thereof, and a device that can be used to administer the active ingredient. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles for such use include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

5.6. Combination Therapy

The methods for treating or preventing diseases, disorders and conditions according to this invention can further comprise administering to the subject an effective amount of one or more additional therapeutic agents. Such therapeutic agents may include those conventionally used to prevent or treat a particular disease, disorder or condition (such as a particular cancer, an autoimmune disease, inflammatory disorder, or other disorder involving PDE4 or elevated levels of cytokines). For example, other therapeutic agents may include, without limitation, steroids, non-steroidal anti-inflammatory agents, antihistamines, analgesics, anti-cancer agents and suitable mixtures thereof. In such combination therapy treatment, both the compounds of this invention and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment of the invention where another therapeutic agent is administered to a subject, the effective amount of the compound of this invention is less than its effective amount when the other therapeutic agent is not administered. In another embodiment, the effective amount of the conventional agent is less than its effective amount when the compound of this invention is not administered. In this way, undesired side effects associated with high doses of either agent may be minimized. Other potential advantages (including without limitation improved dosing regimens and/or reduced drug cost) will be apparent to those of skill in the art.

In the case of autoimmune and inflammatory conditions, the other therapeutic agent can be a steroid or a non-steroidal anti-inflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9^{th} ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Of particular relevance to allergic disorders, the other therapeutic agent can be an anthihistamine. Useful antihistamines include, but are not limited to, loratadine, cetirizine, fexofenadine, desloratadine, diphenhydramine, chlorpheniramine, chlorcyclizine, pyrilamine, promethazine, terfenadine, doxepin, carbinoxamine, clemastine, tripelennamine, brompheniramine, hydroxyzine, cyclizine, meclizine, cyproheptadine, phenindamine, acrivastine, azelastine, levocabastine, and mixtures thereof. For a more detailed description of anthihistamines, see Goodman & Gilman's The Pharmacological Basis of Therapeutics (2001) 651-57, 10^{th} ed).

In the case of cancer, the other therapeutic agent may be selected from any conventional anti-cancer agent appropriate for a target cancer. Examples of such anti-cancer agents include, without limitation, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs that may be used in combination therapy with the compounds of this invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyidinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Examples of anti-cancer therapeutic antibodies that can be used in combination with the compounds of this invention include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-1A cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-V3 integrin antibody (Applied Molecular Evolution/Medimmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zeftyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-antibody (CAT/BASF); CDP870 is a humanized anti-TNF-Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-IgG4 antibody (Celltech); LDP-02 is a humanized anti-47 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-2 antibody (Cambridge Ab Tech). Chemotherapeutic agents that can be used in the combination therapy methods and compositions of the invention include but are not limited to alkylating agents, antimetabolites, natural products, or hormones. Examples of alkylating agents useful for the treatment or prevention of particular cancers (especially those involving T-cell malignancies) include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of treatment or prevention of particular cancers (especially those involving T-cell malignancies) include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of treatment or prevention of particular cancers (especially those involving T-cell malignancies) include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents useful for the treatment or prevention of other cancers in the combination methods and compositions of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of other cancers in the combination methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of other cancers in the combination methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of other cancers in the combination methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other anti-cancer agents that can be used in the combination methods and compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

The compounds of this invention may also be administered in combination with anti-cancer agents which act by arresting cells in the G2-M phases due to stabilized microtubules. In addition to Taxol (paclitaxel), and analogs and derivatives thereof, other examples of anti-cancer agents which act by this mechanism include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261-and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BMBU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IMBE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-0Y-007 (National Health Research Institutes), and SSR-250411 (Sanofi).

In any case where pain in a component of the target disorder, the other therapeutic agent can be an analgesic. Useful analgesics include, but are not limited to, phenacetin, butacetin, acetaminophen, nefopam, acetoamidoquinone, and mixtures thereof.

The foregoing and other useful combination therapies will be understood and appreciated by those of skill in the art. Potential advantages of such combination therapies include the ability to use less of each of the individual active ingredients to minimize toxic side effects, synergistic improvements in efficacy, improved ease of administration or use and/or reduced overall expense of compound preparation or formulation.

5.7. Other Embodiments

The compounds of this invention may be used as research tools (for example, as a positive control to evaluate the mechanism of new TNFa or PDE4 inhibitors by competitive binding assays or to isolate ligands of the compounds of this invention using affinity chromatography. These and other uses and embodiments of the compounds and compositions of this invention will be apparent to those of ordinary skill in the art.

The invention is further defined by reference to the following examples describing in detail the preparation of compounds of the invention. It will be apparent to those skilled in the art that many modifications, both to materials and methods, may be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 6. EXAMPLES

6.1. Synthesis of Intermediates

One embodiment of this invention is a method of preparing intermediates in the synthesis of certain compounds represented by Formula (I). One intermediate is represented by Formula (I_{INT-A}):

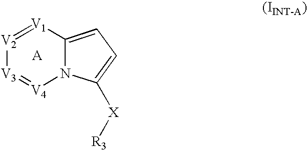

A method for producing I_{INT-A} comprises the step of reacting a Cu^{I} salt with a precursor compound represented by Formula (I_{INT-B}): 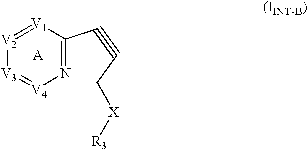
wherein in Formulas (I_{INT-A}) and (I_{INT-B}), Ring A, V₁, V₂, V₃, V₄, X, and R₃ are as described for Formula (I). In this method, R₃ is preferably not a substituted or unsubstituted alkyl group and more preferably, R₃ is a substituted or unsubstituted aryl group.

Additional methods for producing other intermediates are also detailed herein below.

Scheme 1 shows an example of the full synthetic method which incorporates the step of producing a compound of Formula (I_{INT-A}) from a compound of Formula (I_{INT-B}): 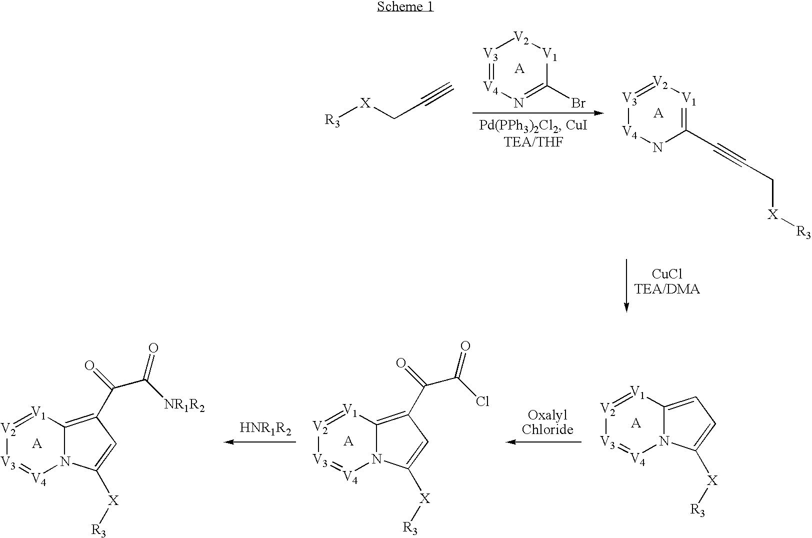
wherein V₁, V₂, V₃, V₄, R₁, R₂, R₃ and X are as defined for compounds of Formula (I).

The intermediate represented by of Formula (I_{INT-A}) is prepared by cyclizing the precursor compound represented by of Formula (I_{INT-B}). The cyclization reaction is carried out in the presence of a Cu^{I} salt such as CuI, CuBr, CuCl, Cu(triflate) and the like. CuCl is the most commonly used CuI salt. Typically, equimolar amounts of the Cu^{I} salt and the precursor compound are used. However, it is also common to use an excess of the CuI salt, for example up to a five fold molar excess, more commonly up to a three fold molar excess, and preferably no more than a 50% molar excess. Suitable solvents for this reaction include polar aprotic solvents such as dimethylacetamide (DMA), dimethylformamide (DMF), dimethylsulfoxide (DMSO), hexamethylphosphoramide (HMPA) and N-methylpyrollidinone (NMO). The reaction is typically carried out at elevated temperatures, e.g., between 70° C. and the boiling point of the solvent, preferably between 100° C. and 160° C. and more preferably between 120° and 140° C. A tertiary amine is typically added to the reaction mixture as a co-solvent, typically in amounts between 1:20 and 4:1 v/v relative to the polar aprotic solvent, more typically between 1:10 and 1:1 v/v. Examples of suitable tertiary amines include triethyl amine, diisopropylethylamine, dimethylaniline, dimethylaminopyridine and the like. Triethylamine is most commonly used. Specific examples of conditions used to carry out this reaction are provided in Example 5.2.5.

The next step in synthesizing the particular compounds of Formula (I) according to Scheme 1 is the acylation of the intermediate represented by Formul (I_{INT-A}) with oxalyl chloride or a synthetic equivalent thereof (e.g., oxalyl bromide). Although equimolar amounts intermediate and acylating agents can be used, typically the acylating agent is used in excess, for example, up to a twenty fold molar excess, preferably up to a ten fold molar excess and more preferably up to a three fold molar excess. Ethereal solvents (e.g., diethyl ether, tetrahydrofuran, 1,4-dioxane, glyme, diglyme and methyl tert-butyl ethyl) and aromatic solvents (e.g., benzene, toluene and xylene) are commonly used. Suitable reaction temperatures range from −50° C. to the boiling point of the solvent and more typically range from −10° C. to room temperature and preferably between −10° C. to 10° C. Specific examples of conditions used to carry out this reaction are provided in Example 5.2.5.

The synthesis according to Scheme 1 is completed by reacting the acylated intermediate with amine HNR₁R₂, wherein R₁ and R₂ are as described for compounds of Formula (I). The acylated intermediate and the amine are mixed in a suitable solvent, e.g., an ethereal solvent or aromatic solvent. Suitable reaction temperatures are as described above for the acylation reaction. Although an excess of one reactant can be used (e.g., up to a ten fold molar excess), more typically between a 20% molar and 100% molar excess. When less than two equivalents of amine HNR₁R₂ are used, a tertiary amine such as triethylamine or dimethylaminopyridine is generally added so that at least two equivalents of amine are present in the reaction mixture relative to the acylated intermediate. Specific examples of conditions used to carry out this reaction are provided in Example 5.2.5.

Scheme 2, shown below, shows a second method for preparing other intermediates useful for producing compounds of Formula (I). In Scheme 2, an intermediate designated (100) is cyclized with a reagent prepared from dimethylformamide and dimethylsulfate, or, alternatively, dimethylformamide di-tert-butylacetal. An exmple of this type of reaction is described more fully in co-pending U.S. Provisional Application entitled “Method for Preparing 3-Acyl-Indolizines,” filed on Sep. 13, 2002, the entire teachings of which are incorporated herein by reference. 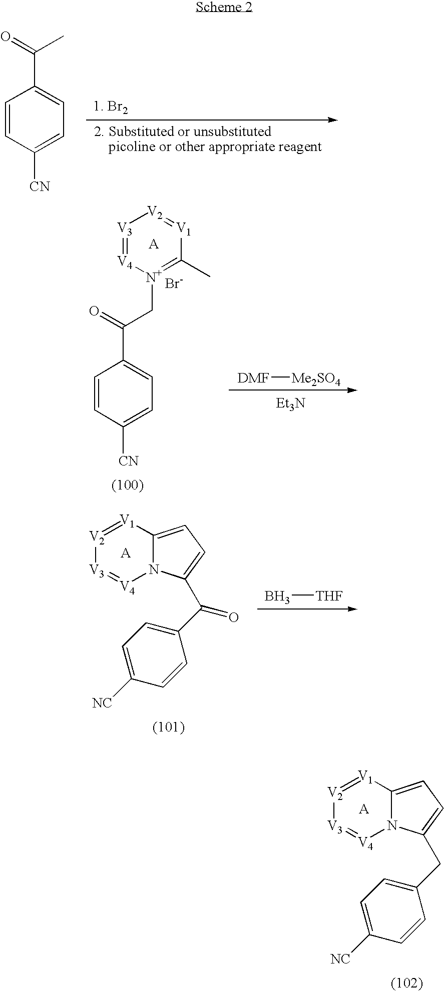
Although the reactions in Scheme 2 are shown with respect to preparing a specific intermediate for producing compounds of Formula (I), other intermediates can be prepared by a suitable selection of starting materials and conditions.

Scheme 3, shown below, shows a third method for preparing additional compounds represented by Formula (I).

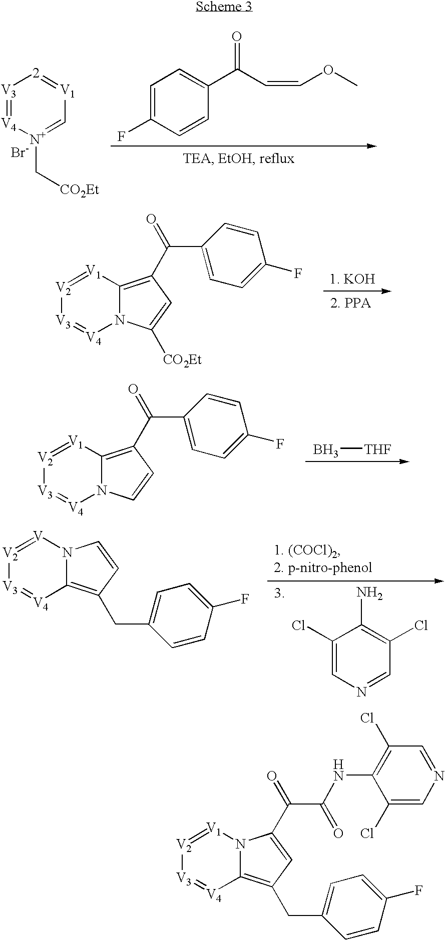

Although the reactions in Scheme 3 are described with respect to preparing a compound having a specific R₂, other compounds of the present invention can be readily prepared by suitable selection of the starting materials and reaction conditions. A specific example of the reaction shown in Scheme 3 is in Example 5.2.1.

6.2. Synthsis of Specific Compounds of Formula (I)

6.2.1. Synthesis of N-(3,5-DICHLORO-PYRIDIN-4-YL)-2-[1-(4-FLUORO-BENZYL)—INDOLIZIN-3-YL]-2-OXO-ACETAMIDE (COMPOUND 1-1)

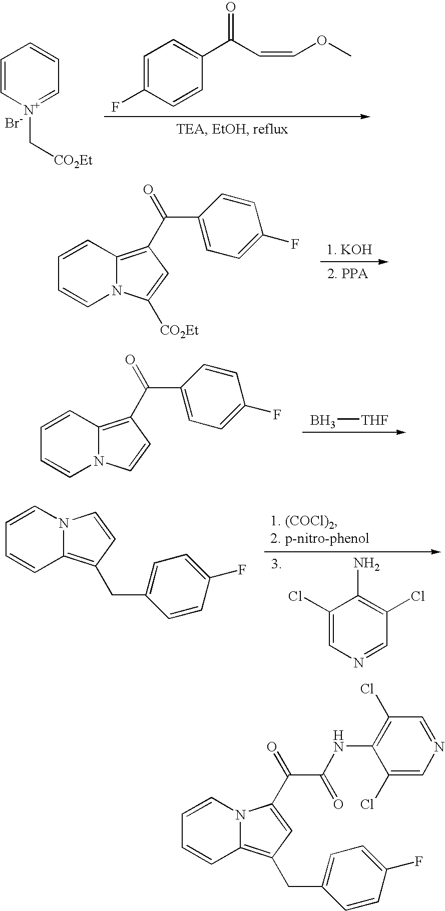

A mixture of 4-fluoroacetophenone (13.81 g, 0.1 mol), methyl formate (7.58 mL, 0.12 mol) and sodium methoxide (made from 2.3 g of Na and 50 mL of methanol) in anhydrous ether (100 mL) was refluxed for 1 hour. The precipitate was filtered out, washed with ether to give 1-(4-fluoro-phenyl)-3-hydroxy-propenone, sodium salt (13.2 g, 70%). It was dissolved then in DMF (120 mL), and dimethyl sulfate (7.7 g, 61 mmol) was added drop-wise to that solution cooled with ice. The resulting mixture was stirred at room temperatufe for 1.5 hour, diluted with water and extracted with ether. Ether extracts were washed with water, 2% NaOH aq, water, and dried over Na₂SO₄. Removal of the solvent and purification on silica gel (30% EtOAc/Hexane) afforded 1-(4-fluoro-phenyl)-3-methoxy-propenone as a solidified oil (7.6 g, 60%). ¹H NMR (CDCl₃): δ 9.60 (d, J=7.2 Hz, 1H), 8.62 (d, J=8.7 Hz, 1H), 7.88 (m, 2H), 7.76 (s, 1H), 7.45 (dd, J=9.0 and 6.9 Hz, 1H), 7.20 (m, 2H), 7.11 (t, J=6.9 Hz, 1H), 4.40 (q, J=7.1 Hz, 2H), 1.40 (t, J=7.0 Hz, 3H).

A solution of 1-(4-fluoro-phenyl)-3-methoxy-propenone (1.03 g, 5.7 mmol), 1-ethoxycarbonylmethylpyridinium bromide (1.4 g, 5.7 mmol) and thriethylamine (1.7 mL, 12.2 mmol) in ethanol (55 mL) was refluxed for 8 hours. After removal of the solvent and excess of triethylamine water was added to the residue and extracted with ethyl acetate. The combined extracts were dried (Na₂SO₄), concentrated and chromatographed on silica gel (16% EtOAc/Hexane) to yield 1-(4-fluoro-benzoyl)-indolizine-3-carboxylic acid ethyl ester (0.75 g, 2.4 mmol, 42%). ¹H NMR (CDCl₃): δ 9.60 (d, J=7.2 Hz, 1H), 8.62 (d, J=8.7 Hz, 1H), 7.88 (m, 2H), 7.76 (s, 1H), 7.45 (dd, J=9.0 and 6.9 Hz, 1H), 7.20 (m, 2H), 7.11 (t, J=6.9 Hz, 1H), 4.40 (q, J=7.1 Hz, 2H), 1.40 (t, J=7.0 Hz, 3H).

1-(4-Fluoro-benzoyl)-indolizine-3-carboxylic acid ethyl ester was refluxed with potassium hydroxide (0.74 g, 12 mmol) in methanol (7 mL) for 3 hours. Removal of the solvent and acidification of the residue with 6N HCL yielded precipitated indolizine-acid which was washed with water, dried and rubbed with polyphosphoric acid. The resulting paste was heated at 100° C. for 1 hour, poured into ice-water, neutralized with sodium hydroxide solution and extracted with ethyl acetate. The combined extracts were dried (Na₂SO₄), concentrated and chromatographed on silica gel (16% EtOAc/Hexane) to afford (4-fluoro-phenyl)-indolizine-1-yl-methanone (0.2 g, 35%). (4-fluoro-phenyl)-indolizine-1-yl-methanone was dissolved in THF (5 mL) and treated with 1 M solution of BH₃-THF (1.75 mL). The reaction mixture was stirred for 45 min, cooled with ice and quenched carefully with ice-water. Ethyl acetate was added and the organic layer was separated, dried (Na₂SO₄), concentrated and chromatographed rapidly on silica gel (10% EtOAc/Hexane) to afford 1-(4-fluoro-benzyl)-indolizine (0.16 g, 85%). ¹H NMR (CDCl₃): δ 7.83 (d, J=6.9 Hz, 1H), 7.31 (d, J=8.9 Hz, 1H), 7.20 (m, 2H), 6.93 (m, 2H), 6.55 (m, 2H), 6.34 (m, 2H), 4.07 (s, 2H).

To a stirred solution of oxalyl chloride (0.025 mL, 0.28 mmol) in dichloromethane (4 mL) cooled with ice a solution of 1-(4-fluoro-benzyl)-indolizine (52 mg, 0.23 mmol) in dichloromethane (4 mL) was added drop-wise and the reaction mixture was stirred at 0° C. for 10 min. Then 4-nitro-phenol (64.2 mg, 0.46 mmol) was added as a solid and the reaction mixture was stirred at r.t. for 15 min, treated with triethylamine (0.08 ml, 0.56 mmol), filtered through a layer of silica gel and eluated with dichloromethane until no yellow colored washings were observed. The resulting solution was concentrated and dissolved in anhydrous DMF (0.5 mL). To a solution of 4-amino-3,5-dichloro-pyridine (75.2 mg, 0.46 mmol) in anhydrous DMF (1 mL) NaH (20.3 mg, 0.5 mmol) was added under nitrogen purge at 0° C., the resulted mixture was stirred for 5 min and combined with indolizine solution. The reaction mixture was stirred ar r.t. for 30 min, quenched with water and extracted with ethyl acetate. The ethyl acetate solution was washed with water (4 times), brine, dried (Na₂SO₄), and the concentrated residue was purified on silica gel (16% EtOAc/Hexane) to afford N-(3,5-dichloro-pyridin4-yl)-2-[1-(4-fluoro-benzyl)-indolizin-3-yl]-2-oxo-acetamide (30 mg, 29%) as a yellow crystalline. ¹H NMR (CDCl₃): δ 10.04 (d, J=7.2 Hz, 1H), 9.55 (brs, 1H), 8.58 (s, 2H), 8.42 (s, 1H), 7.50 (d, J=8.4 Hz, 1H), 7.36 (t, J=7.2 Hz, 1H), 7.19 (m, 2H), 7.09 (t, J=6.9 Hz, 1H), 6.96 (m, 2H), 4.10 (s, 2H); ESMS clcd for C₂₂H₁₄Cl₂FN₃O₂: 441.04; Found: 442.0 (M+H)⁺.

6.2.2. Synthesis of 2-[1-(4-FLUORO-BENZYL)-INDOLIZIN-3-YL]-N-(3-METHYL-ISOTHIAZOL-5-YL)-2-OXO-ACETAMIDE (COMPOUND 1-2)

To a stirred solution of oxalyl chloride (0.025 mL, 0.28 mmol) in anhydrous THF (4 mL) cooled with ice a solution of 1-(4-fluoro-benzyl)-indolizine (52 mg, 0.23 mmol) in anhydrous THF (4 mL) was added drop-wise and the reaction mixture was stirred at 0° C. for 10 min. A solution of 5-amino-3-methylisothiazole (64 mg, 0.56 mmol) in anhydrous THF (4 mL) was added then and the reaction mixture was stirred at r.t. for 1 hour. Water and ethyl acetate were added, organic layer was washed with water, brine, dried and purified on silica gel to yield 2-[1-(4-fluoro-benzyl)-indolizin-3-yl]-N-(3-methyl-isothiazol-5-yl)-2-oxo-acetamide (36 mg, 40%) as a yellow crystalline. ¹H NMR (CDCl₃): δ 10.49 (brs, 1H), 10.01 (d, J=6.9 Hz, 1H), 8.53 (s, 1H), 7.50 (dtr, J=8.9 and 1.2 Hz, 1H), 7.36 (td, J=7.9 and 1.2 Hz, 1H), 7.19 (m, 2H), 7.08 (td, J=6.9 and 1.2 Hz, 1H), 6.97 (m, 2H), 6.78 (s, 1H), 4.10 (s, 2H), 2.46 (s, 3H); ESMS clcd for C₂₁H₁₆FN₃O₂S: 393.09; Found: 394.0 (M+H)⁺.

6.2.3. Synthesis of 2-[1-(4-FLUORO-BENZYL)-INDOLIZIN-3-YL]-2-OXO-N-PYRIDIN-3-YL-ACETAMIDE (COMPOUND 1-3)

2-[1-(4-Fluoro-benzyl)-indolizin-3-yl]-2-oxo- N-pyridin-3-yl-acetamide (23 mg, 39%) was prepared as described above using 3-aminopyridine (53 mg, 0.56 mmol). ¹H NMR (CDCl₃): δ10.02 (d, J=7.2 Hz, 1H), 9.59 (brs, 1H), 8.80 (d, J=2.4 Hz, 1H), 8.50 (s, 1H), 8.42 (d, J=3.6 Hz, 1H), 8.32 (d, J=8.4 Hz, 1H), 7.50 (d, J=8.4 Hz, 1H), 7.34 (m, 2H), 7.20 (m, 2H), 7.08 (t, J=6.9 Hz, 1H), 6.97 (m, 2H), 4.11 (s, 2H); ESMS clcd for C₂₂H₁₆FN₃O₂: 373.12; Found: 374.0 (M+H)⁺.

6.2.4. Synthesis of N-(3,5-DICHLORO-1-OXY-PYRIDIN-4-YL)-2-[7-(4-FLUORO-BENZYL)-PYRROLO[1,2-B] PYRIDAZI N-5-YL]-2-OXO-ACETAMIDE (COMPOUND 1-4) AND RELATED COMPOUNDS

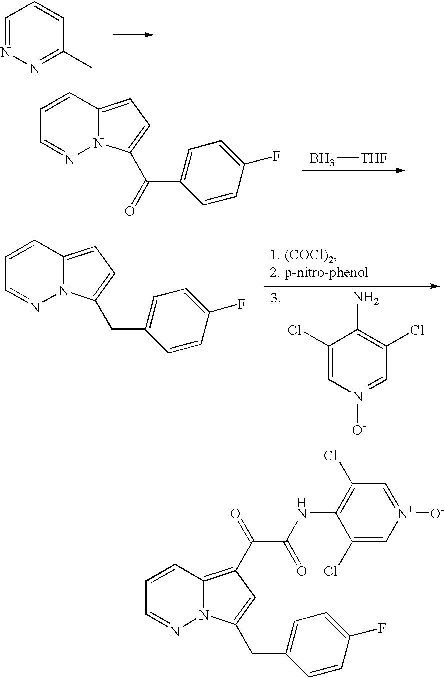

To a 2-bromo-4′-fluoroacetophenone (2.2 g, 10 mol) CH₃CN (5 ml) solution, was added 3-methylpyridazine (1.2 g, 10 mmol), stirred overnight at rt, and to the mixture was added EtOAc-Hexanes (1:1, 20 ml), the precipitate was collected by filtration and washed with EtOAc, and directly used for the next step. To the resulting bromide salt (2 g) a DMF (10 ml) suspension solution was added DMF-Me₂SO₄ (14 ml, the mixture obtained by stirring and keep a mixture of 1 eq. DMF and eq Me₂SO₄ at 60-80° C. for 3 h, then rt), and stirred at rt for 15 min., then to the mixture was added Et₃N (15 ml)and stirred for 2 hr keeping inner temp. at 25-40° C. To the resulting mixture was added ice water (30 ml), extracted with EtOAc(100 ml), washed with water (20 ml×3), dried with Na₂SO₄. After evaporation of the solvents, the residue was subjected to silica gel CC (Hexane: CH₂Cl₂ 1:1, CH₂Cl₂), to give (4-fluoro-phenyl)-pyrrolo[1,2-b]pyridazin-7-yl-methanone (210 mg)

To (4-fluoro-phenyl)-pyrrolo[1,2-b]pyridazin-7-yl-methanone (210 mg, 0.88 mmol) THF (10 ml) solution was added BH₃-THF(1 M, 2.5 ml), the resulting mixture was stirred at rt for 1 hr. The reaction was quenched with ice water (10 ml), and the mixture was extracted with EtOAc (50 ml), dried with Na₂SO₄. The solvents was evaporated, and the residue was subjected to silica gel CC (Hexane:CH₂Cl₂ 1:1, CH₂Cl₂), to give 7-(4-fluoro-phenyl)-pyrrolo[1,2-b]pyridazine (100 mg, yield 50%)

To oxalyl chloride (64 mg, 0.5 mmol) CH₂Cl₂ solution (2 ml), was added 7-(4-fluoro-phenyl)-pyrrolo[1,2-b]pyridazine (100 mg) CH₂Cl₂ solution (1 ml) at 0° C., and stirred for 5 min, to the mixture was added 4-nitrophenol (139 mg, 1 mmol), and stirred for another 5 min. at rt, TEA (150 mg) was added, and the resulting solution was filtered with a short silica gel funnel immediately, and washed with CH₂Cl₂ (15 ml). The CH₂Cl₂ solvent was evaporated and the residue was mixed with NaH-4-amino-3,5-dichloropyridine N-oxide DMF solution (prepared from 40 mg 60% NaH, 180 mg N-oxide in 8 ml DMF) at rt, stirred for 20 min., quenched with 1% acetic acid in water (15 ml). The mixture was extracted with EtOAc (30 ml) and washed with water (10 ml×3). After removal of the solvent, the residue was subjected to silica gel CC(CH₂Cl₂, Hexanes:EtOAc 1:1, EtOAc), and preparative TLC (MeOH: CH₃Cl: NH₄OH, 7: 92: 1), to give N-(3,5-dichloro-1-hydroxy-pyridin-4-yl)-2-[7-(4-fluoro-benzyl)-pyrrolo[1,2-b]pyridazin-5-yl]-2-oxo-acetamide. (3 mg, yield: 1.5%). ¹H-NMR (CDCl₃) δ 4.24 (s, 2H), 6.86-7.29 (m, 5H), 7.95 (s, 1H), 8.27 (s, 2H), 8.41 (m, 1H), 8.81 (m, 1H), 9.07(s, 1H)ppm. ESMS calcd for C₂₁H₁₄Cl₂FN₄O₃: 459.04; Found: 460.1 (M+H)⁺.

2-[7-(4-Cyano-benzyl)-pyrrolo[1,2-b]pyridazin-5-yl]-N-(3,5-dichloro-pyridin-4-yl)-2-oxo-acetamide (Compound 1-5), 2-[7-(4-Methoxy-benzyl)-pyrrolo[1,2-b]pyridazin-5-yl]-2-oxo-N-pyridin4-yl-acetamide (Compound 1-6), 2-[7-(4-Chloro-benzyl)-pyrrolo[1,2-b]pyridazin-5-yl]-N-isoxazol-5-yl-2-oxo-acetamide (Compound 1-7), and N-(3,5-Dichloro-pyridin4-yl)-2-[6-(4-methoxy-benzyl)-pyrrolo[1,2-a]pyrazin-8-yl]-2-oxo-acetamide (Compound 1-8), N-(3,5-Dichloro-pyridin4-yl)-2-[7-(4-fluoro-benzyl)-pyrrolo[1,2-c]pyrimidin-5-yl]-2-oxo-acetamide (Compound 1-9) can be synthesized by a route analogous to the synthesis for Compound 14 shown above.

6.2.5. Synthesis of 2-[5-(4-CYANO-BENZYL)-PYRROLO[2,1-B]THIAZOL-7-YL]-N-(3-ME THYL-ISOTHIAZOL-5-YL)-2-OXO-ACETAMIDE (Compound I-10) and Related Compounds

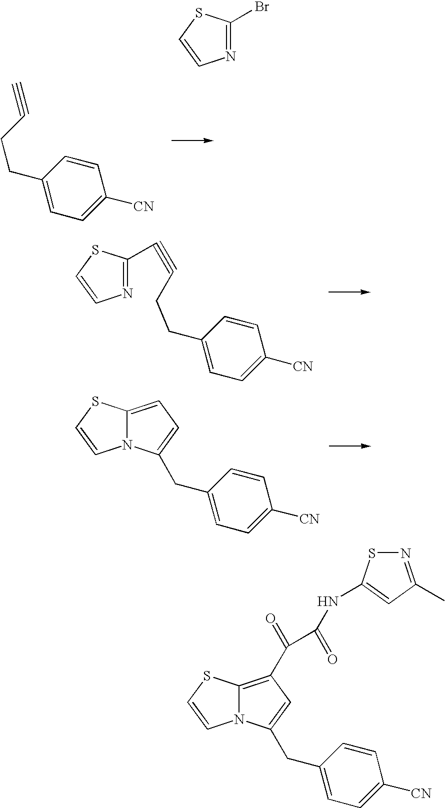

To a stirred suspension of 2-bromothiazole (160 mg, 1 mmol), dichlorobis(triphenylphosphine)palladium (II) (14 mg, 0.02 mmol) and Copper (I) iodide (3.8 mg, 0.02 mmol) in degassed TEA (5 mL) was added 4-But-3-ynyl-benzonitrile (155 mg, 1 mmol). The mixture was then heated to 60° C. and stirred under an atmosphere of dry N₂ for 6 h. Undissolved material was filtered off and the filtrate was concentrated followed by SGC purification (hexane to 10% EtOAc/Hexane).

4-(4-Thiazol-2-yl-but-3-ynyl)-benzonitrile was obtained as a white powder (140 mg, 59% yield).

A mixture of 4-(4-Thiazol-2-yl-but-3-ynyl)-benzonitrile (110 mg, 0.46 mmol) and copper (I) chloride (23 mg, 0.23 mmol) in N,N-dimethyl acetamide (2.1 mL) and TEA (0.29 mL) was stirred at 130° C. under N₂ for 21 h. After being cooled to rt the mixture was filtered through celite and was then portioned between EtOAc (15 mL) and H₂O (10 mL). EtOAc layer was separated and washed with water (2 times with 10 mL each), dried with Na₂SO₄. Removal of solvent followed by SGC (hexane to 2% EtOAc/Hexane) afforded the product 4-Pyrrolo[2,1-b]thiazol-5-yl-methyl-benzonitrile as a yellow solid (58 mg, 53% yield). ¹H-NMR (CDCl₃) δ 4.1 (s, 2H), 6.1 (d, 1H, J=3.6), 6.5 (d, 1H, J=5), 6.55 (d, 1H, J=4.2), 6.85 (d, J=4.2), 7.41 (d, 2H, J=9), 7.51 (d, 2H, J=9)ppm. ESMS calcd for C₁₄H₁₀N₂S: 238.1; Found: 239.1 (M+H)⁺.

A solution of 4-Pyrrolo[2,1-b]thiazol-5-yl-methyl-benzonitrile (28 mg, 0.12 mmol) in dry THF (1 mL) was added slowly to a stirred solution of oxalyl chloride (12.3 uL, 0.14 mmol) in dry THF at 0° C. After 30 min stirring at the same temperature, the volatile components were removed under reduced pressure and dried in vacuo. The residue was then dissolved in dry THF (1 mL) at 0° C., a solution of 3-Methyl-isothiazol-5-ylamine (16 mg, 0.14 mmol) in dry THF (1 mL) was added through a syringe. After 2 h stirring at rt, EtOAc (20 mL) was added, washed with H₂O (2×15 mL) and brine (15 mL), dried (Na₂SO₄). Removal of solvent provide a red solid which was washed with EtOAc.

2-[5-(4-Cyano-benzyl)-pyrrolo[2,1-b]thiazol-7-yl]-N-(3-methyl-isothiazol-5-yl)-2-oxo-acetamide was obtained as a red crystalline (20 mg, 42% yield). ¹H-NMR (DMSO-d₆) δ ppm: 2.3 (s, 3H), 4.3 (s, 2H), 7.1 (s, 1H), 7.25 (d, 1H, J=4), 7.45 (d, 1H, J=4), 7.58 (d, 2H, J=9), 7.80 (d, 2H, J=9), 8.05 (s, 1H), 12.6 (b, 1H, NH). ESMS calcd for C₂₀H₁₄N₄S₂: 406.1; Found: 407.1 (M+H)⁺.

2-[5-(4-Cyano-benzyl)-pyrrolo[2,1-b]oxazol-7-yl]-N-(3-methyl-isothiazol-5-yl)-2-oxo-acetamide (Compound I-11), 2-[5-(4-Cyano-benzyl)-pyrrolo[2,1-b]thiazol-7-yl]-N-(3,5-dichloro-pyridin4-yl)-2-oxo-acetamide (Compound 1-12), N-(3,5-Dichloro-1-oxy-pyridin-4-yl)-2-[5-(4-fluoro-benzyl)-pyrrolo[2,1-b]thiazol-7-yl]-2-oxo-acetamide (Compound 1-13) and 2-[5-(4-Cyano-benzyl)-1-methyl-H-pyrrolo[1,2-a]imidazol-7-yl]-N-(3-methyl-isothiazol-5-yl)-2-oxo-acetamide (Compound I-14) can be synthesized by a route analogous to the synthesis for Compound I-10 shown above.

6.3. In-Vitro Assay Measuring TNFα Inhibition

Reagents. Lipopolysaccharide (LPS, Serratia marscencens) was obtained from Sigma (St. Louis, Mo.). RPMI-1640 medium and fetal calf serum (FCS) were purchased from the ATCC (Manassas, Va.).

Assay. Human peripheral blood cells (PBMC) were isolated by centrifugation using Ficoll-Paque (Pharmacia Biotech, Uppsala, Sweden) and suspended in RPMI-1640 medium supplemented with 10% FCS, 100 U/mL penicillin, and 100 μg/mL streptomycin. The cells were then plated in the wells of a 96-well plate at a concentration of 5×105 cells/well, and stimulated by adding LPS (1 μg/mL). Each test compound was dissolved in DMSO and added to the wells. The final DMSO concentration was adjusted to 0.25% in all cultures, including the compound-free control, and the concentrations of each test compound ranged from 0 to 10 μM. Cell-free supernatants were taken 18 h later for measurement of cytokines. Cell viability was assessed using the bioreduction of MTS [3-(4,5-dimethylthiazol-2-yl)-5-(3-carboxymethoxyphenyl)-2-(4-sulophenyl)-2H-tetrazolium] after 18 h and 48 h. Cell survival was estimated by determining the ratio of the absorbance in each of the compound-treated cultures to that in the compound-free control.

The supernatant was assayed for the amount of TNFα by using an ELISA assay with anti-human TNFa antibodies (Cell Sciences, Norwood, Mass.). The assay was carried out following the manufacturer's instructions.

Compounds I-4 and I-10 were tested. These compounds demonstrated IC₅₀ values of about 50 nM and 1 μM, respectively. The results of this experiment demonstrate that the tested compounds of this invention inhibit TNFa production.

6.4. In-Vitro Assay Measuring PDE4 Inhibition

PDE4 was prepared from U937 human monocytic cells according to the method of Tenor et al. (Clin Exp Allegy (1995) 25:625-633). Briefly, U937 cells were homogenized in a mixture of pH 7.4 containing 10 mM Hepes, 1 mM b-mercaptoethanol, 1 mM MgCl₂, 1 mM EGTA, 137 mM NaCl, 2.7 mM KCl, 1.5 mM KH₂PO₄, 8.1 mM Na₂HPO₄, 5 μM pepstain A, 10 μM leupeptin, 50 μM PMSF, 10 μM soybean trypsin inhibitor, and 2 mM benzamindine. The homogenate was centrifuged at 200,000×g for 30 min. PDE4 activity in the supernatant was assayed in a 200 μl reaction containing 40 mM Tris-HCl, pH 7.5, 23 nM [³H]-adenosine 3′,5′ cyclic monophosphate (cAMP), 8.3 mM MgCl₂, 1.7 mM EGTA, 0.25% DMSO, and a testing compound. The assay mixture was incubated at 37° C. for 30 min and the reaction was terminated by the addition of 100 μl of yttrium silicate SPA beads (Amersham Pharmacia Biotech, Piscataway, N.J.) suspended in 18 mM ZnSO₄. The assay mixture was rotated for 3 min to ensure the binding of [³H]-5′adenosine monophosphate to the beads. Finally, the beads was spun down, washed twice with 6 mM ZnSO₄, resuspended in 100 μl of 0.1 N NaOH, and then counted for radioactivity in a liquid scintillation counter.

Compound I-4 was tested. This compound showed an IC₅₀ value of about 5 nM. The results of this experiment demonstrate that the tested compounds of this invention inhibit PDE4 production.

6.5. In-Vitro Assay Measuring Anti-Cancer Activity

In vitro anti-cancer activity was determined in human cancer cell line MDA435 (human breast cancer), obtained from ATCC (American Type of Culture Collection).

The cell line was maintained in RPMI1640(GIBCO) supplemented with 10% FCS, 100 units/ml penicillin, 100 ug/ml streptomycin, and 2 mM L-glutamine. The cells were split every third day and diluted to a concentration of 2×10⁵ cells/ml one day before experiment. The experiment was performed on exponentially growing cell culture. Cell densities were 2.5×104 cells/ml in this experiment.

A stock solution of Compound (I-4) was prepared by dissolving the compound at a concentration of 1 mM in 100% DMSO. Final concentrations were obtained by diluting the stock solution directly into the tissue culture medium. Cells were incubated with varying concentrations of the compound for 72 hours and the IC₅₀ was determined by MTS (i.e. 3-(4.5.-dimethylthiazol-2-yl)-2.5-diphenyl tetrazolium bromide) assay. In this experiment, IC₅₀ stands for the concentration of compound required to inhibit 50% tumor cell growth. Compound (I-4) provided an IC₅₀ value of about 1 μM.

All publications, patent applications, patents, and other documents cited herein are incorporated by reference in their entirety. In case of conflict, the present specification, including definitions, will control. In addition, the materials, methods, and examples are illustrative only and not intended to be limiting.

1. A compound of Formula (I): 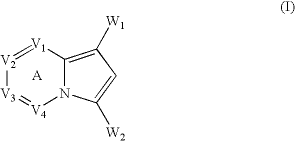
wherein:
one of W₁ and W₂ is 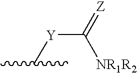
and the other is 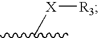
V₁, V₂, V₃ and V₄ are independently CR₆ or N; or alternatively, V₁ and V₂ taken together or V₃ and V₄ taken together may be replaced with S, O, or NR₇ to form a fused 5-membered heterocyclic ring, and wherein two adjacent positions on Ring A may optionally be joined to create a fused aryl group, provided that when W₁ is 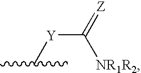
V₁, V₂, V₃ and V₄ may not all be CR₆;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
Y is —C(R₄R₅)—, —N(R₄)—, —O—, —S—, —S(O)—, —S(O)₂—, —C(═O)—, —C(═S)—, —C(═O)—N(R₄)—, —C(═N—OR₈)—, —C(═N—R₈)—, or —N(R₄)—C(═O)—;
Z is ═O, ═S, ═N—OR₈ or ═NR₈;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, or alternatively, NR₁R₂, taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group;
each R₄ and R₅ is independently —H or a substituted or unsubstituted aliphatic group;
each R₆ is independently —H or a Ring A substituent;
each R₇ is independently —H or a heteroaryl ring nitrogen substituent and each R₈ is independently —H, an unsubstituted aliphatic group, a substituted aliphatic group, an unsubstituted non-aromatic heterocylic group, a substituted non-aromatic heterocylic group, an unsubstituted aryl group, or a substituted aryl group;
and pharmaceutically acceptable salts and prodrugs thereof.

2. The compound according to claim 1, wherein
X is —C(R₄R₅)—, —N(R₄)—, —C(═O)— or —O—;
Y is —C(R₄R₅)— or C═O;
Z is ═O;
R₁ is —H;
R₂ is a substituted or unsubstituted alkyl group or a substituted or unsubstituted aryl group;
R₃ is a substituted or unsubstituted aryl group;
R₆ is independently selected from H, halo, —C₁-C₄ alkyl, —C₁-C₄ alkoxy, —C₁-C₄ haloalkyl, C₁-C₄ haloalkoxy, —C₁-C₄ acyl, amido, substituted amido, —NO₂, —CN, —OH, —NH₂ and substituted amino; and
each R₈ is independently —H or a substituted or unsubstituted aliphatic group.

3. The compound according to claim 2, wherein:
X is —CH₂—, —CH(lower alkyl)-, —NH—, —N(lower alkyl)-, —C(═O)— or —O—;
Y is C═O;
R₂ is an unsubstituted aryl group or an aryl group substituted with lower alkyl, amido, cyano or halo;
R₃ is a substituted or unsubstituted phenyl, pyridyl or thienyl group;
R₄ and R₅ are both H; and
each R₈ is independently —H or a substituted or unsubstituted lower alkyl.

4. The compound according to claim 1, having the structure of Formula (Ia), (Ib), (Ic), (Id), (Ie), (If) or (Ig): 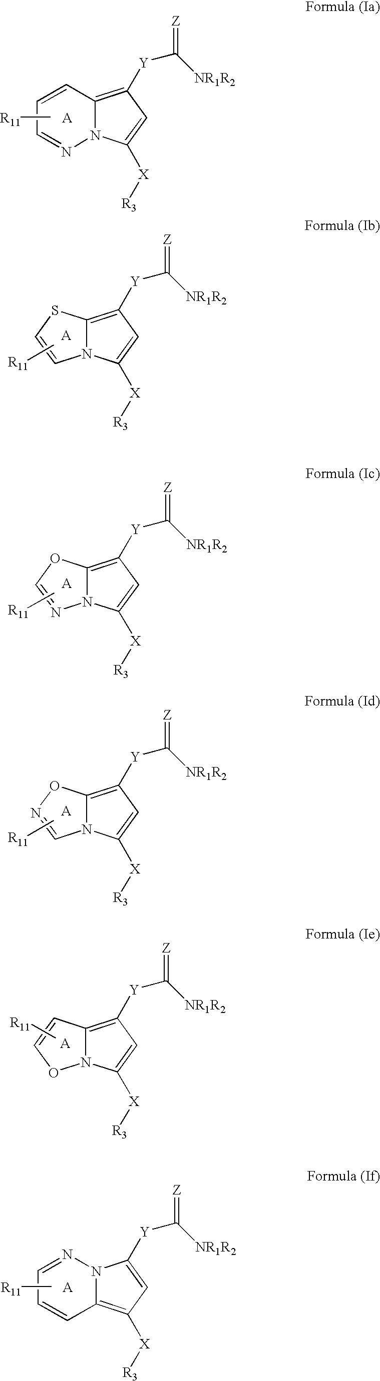 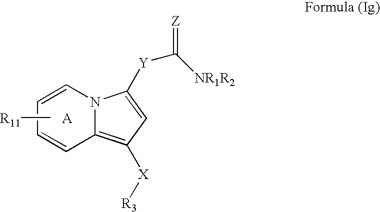
wherein X is a covalent bond, —C(R₄R₅)—, —N(R₄)—, —O—, —S—, —S(O)—, —S(O)₂—, —C(═O)—, —C(═O)—N(R₄)—C(═O)—;
Y is —C(R₄R₅)—, —N(R₄)—, —O—, —S—, —S(O)—, —S(O)₂—, —C(═O)—, —C(═S)—, —C(═O)—N(R₄)—, —C(═N—OR₈)—, —C(═N—R₈)—, or —N(R₄)—C(═O)—;
Z is ═O, ═S, ═N—OR₈ or ═NR₈;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group; or alternatively, NR₁R₂, taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group;
each R₄ and R₅ is independently —H or a substituted or unsubstituted aliphatic group;
each R₈ is independently —H, an unsubstituted aliphatic group, a substituted aliphatic group, an unsubstituted non-aromatic heterocylic group, a substituted non-aromatic heterocylic group, an unsubstituted aryl group, or a substituted aryl group;
each R₁₁ is independently selected from Ring A substituents (preferably, selected from the group consisting of H, hydroxyl, cyano, nitro, halo, a substituted or unsubstituted amino group, a substituted or unsubstituted acyl group, a substituted or unsubstituted amido group, a substituted or unsubstituted alkyl group, a substituted or unsubstituted alkoxy group, or a substituted or unsubstituted aryl group; and
pharmaceutically acceptable salts and prodrugs thereof.

5. The compound according to claim 4, wherein X is —C(R₄R₅)—, —N(R₄)—, —C(═O)— or —O—;
Y is —C(R₄R₅)— or C═O;
Z is ═O;
R₁ is —H;
R₂ is a substituted or unsubstituted alkyl group or a substituted or unsubstituted aryl group;
R₃ is a substituted or unsubstituted aryl group; and
each R₈ is independently —H or a substituted or unsubstituted aliphatic group.

6. The compound according to claim 3, wherein:
X is —CH₂—, —CH(lower alkyl)-, —NH—, —N(lower alkyl)-, —C(═O)— or —O—;
Y is C═O;
R₂ is an unsubstituted aryl group or an aryl group substituted with lower alkyl, amido, cyano or halo;
R₃ is a substituted or unsubstituted phenyl, pyridyl or thienyl group;
R₄ and R₅ are both H; and
each R₈ is independently —H or a substituted or unsubstituted lower alkyl.

7. The compound according to claim 1, wherein R₂ is selected from the group consisting of Formulas (II)-(XV): 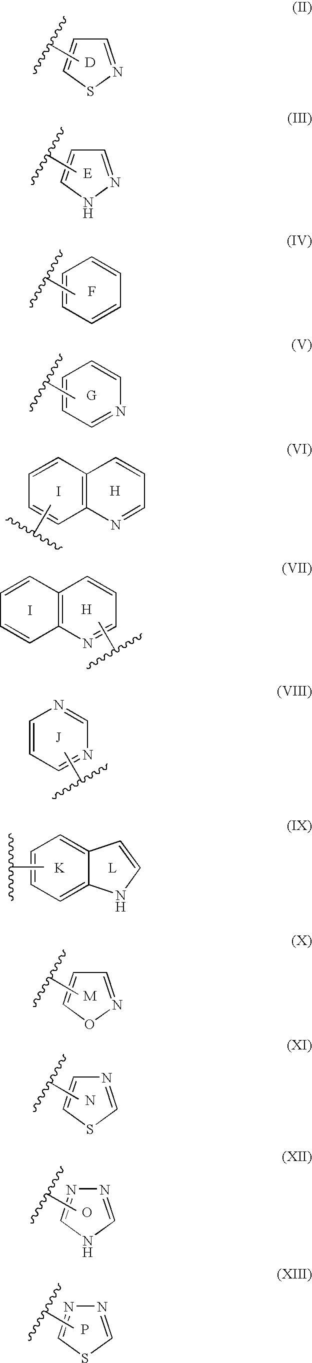 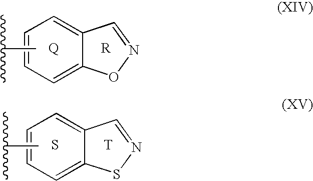
wherein each of rings Rings D-T may be substituted or unsubstituted.

8. The compound according to claim 4, wherein R₂ is selected from the group consisting of Formulas (II)-(XV): 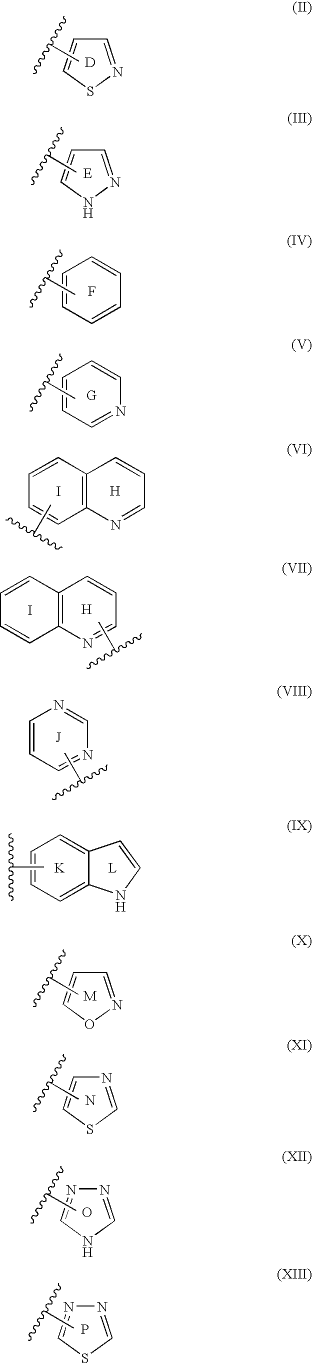 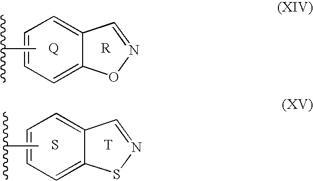
wherein each of rings Rings D-T may be substituted or unsubstituted.

9. The compound according to claim 7, wherein R₂ is selected from Formulas (XVI)-(XXI): 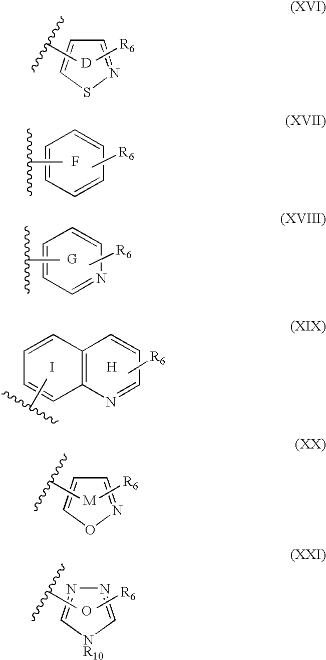
wherein
each R₆ is independently selected from the group consisting of H, hydroxyl, cyano, nitro, halo, a substituted or unsubstituted alkyl group, a substituted or unsubstituted alkoxy group, or a substituted or unsubstituted aryl group; and
R₁₀ is —H or a substituted or unsubstituted alkyl group.

10. The compound according to claim 8, wherein R₂ is selected from Formulas (XVI)-(XXI): 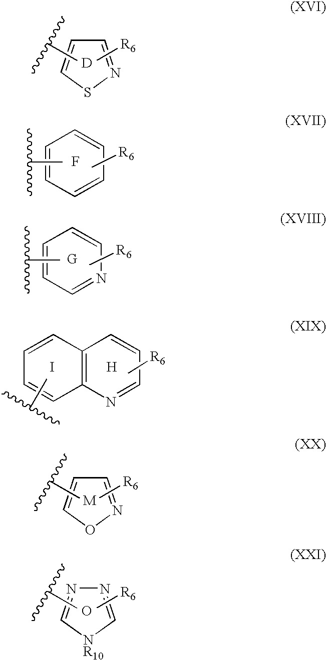
wherein
each R₆ is independently selected from the group consisting of H, hydroxyl, cyano, nitro, halo, a substituted or unsubstituted alkyl group, a substituted or unsubstituted alkoxy group, or a substituted or unsubstituted aryl group; and
R₁₀ is —H or a substituted or unsubstituted alkyl group.

11. The compound according to claim 9, wherein R₂ is selected from Formulas (XXII)-(XXVII): 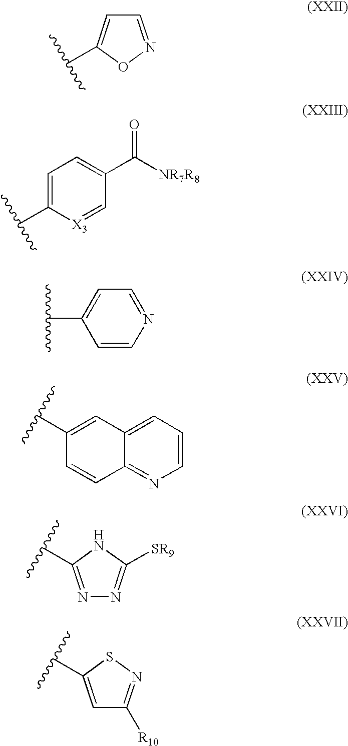
wherein X₃ is —CH— or —N—;
R₇ and R₈ are independently —H or an alkyl group or alternatively, —NR₇R₈, taken together, is a nitrogen-containing non-aromatic heterocyclic group;
R₉ is an alkyl group; and
R₁₀ is —H or an alkyl group.

12. The compound according to claim 10, wherein R₂ is selected from Formulas (XXII)-(XXVII): 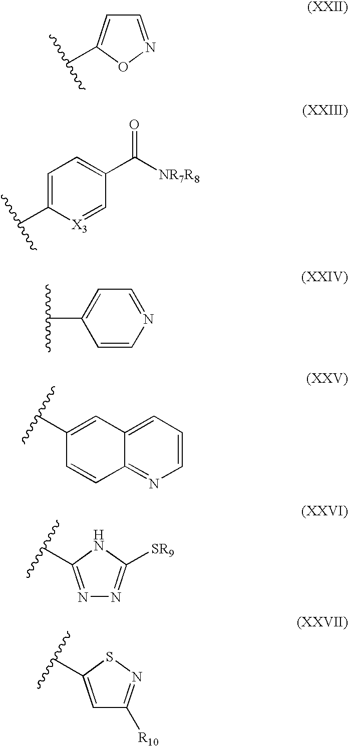
wherein X₃ is —CH— or —N—;
R₇ and R₈ are independently —H or an alkyl group or alternatively, —NR₇R₈, taken together, is a nitrogen-containing non-aromatic heterocyclic group;
R₉ is an alkyl group; and
R₁₀ is —H or an alkyl group.

13. A compound selected from the group consisting of Compounds (I-1) through (I-14).

14. A pharmaceutical composition comprising at least one compound according to claim 1 and a pharmaceutically acceptable carrier.

15. The pharmaceutical composition of claim 14, further comprising one or more additional therapeutic agents.

16. The pharmaceutical composition of claim 15, wherein the additional therapeutic agent is an agent against cancer agent, an autoimmune disease, an inflammatory disorder or pain.

17. A method for treating cancer, an inflammatory disorder or an autoimmune disease comprising the step of administering to a subject in need thereof an effective amount of the pharmaceutical composition according to claim 14.

18. A method for preventing cancer, an inflammatory disorder or an autoimmune disease comprising the step of administering to a subject in need thereof an effective amount of the pharmaceutical composition according to claim 14.

19. A method for preventing or treating a disorder involving PDE4 or elevated levels of cytokines comprising the step of administering to a subject in need thereof an effective amount of the pharmaceutical composition according to claim 14.

20. The method according to claim 19, wherein the disorder is characterized, mediated or exacerbated by overproduction or activity of TNFα.

21. The method according to claim 19, wherein the disorder is characterized, mediated or exacerbated by overproduction or activity of PDE4.

22. A method of inhibiting TNFα or PDE4 in a cell comprising the step of contacting the cell with an effective amount of a compound according to claim 1.

23. A method for reducing TNFα levels in a subject comprising administering to the subject an effective amount of a compound according to claim 1.

24. A method for suppressing inflammatory cell activation comprising the step of contacting the cell with an effective amount of a compound according to claim 1.

25. A method of preparing a compound of Formula (I_{INT-A}): 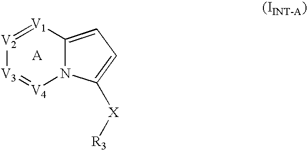
comprising the step of reacting a Cu^{I} salt with a precursor compound represented by Formula (I_{INT-B}): 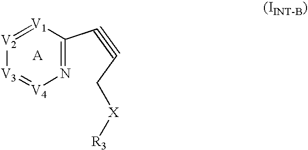
wherein
V₁, V₂, V₃ and V₄ are independently CR₆ or N; or alternatively, V₁ and V₂ taken together or V₃ and V₄ taken together may be replaced with S, O, or NR₇ to form a fused 5-membered heterocyclic ring, and wherein two adjacent positions on Ring A may optionally be joined to create a fused aryl group, provided that when W₁ is 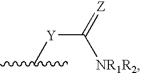
V₁, V₂, V₃ and V₄ may not all be CR₆;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
Y is —C(R₄R₅)—, —N(R₄)—, —O—, —S—, —S(O)—, —S(O)₂—, —C(═O)—, —C(═S)—, —C(═O)—N(R₄)—, —C(═N—OR₈)—, —C(═N—R₈)—, or —N(R₄)—C(═O)—;
Z is ═O, ═S, ═N—OR₈ or ═NR₈;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group;
or alternatively, NR₁R₂, taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group, provided that R₃ is not a substituted or unsubstituted alkyl group;
each R₄ and R₅ is independently —H or a substituted or unsubstituted aliphatic group;
each R₆ is independently —H or a Ring A substituent;
each R₇ is independently —H or a heteroaryl ring nitrogen substituent; and
each R₈ is independently —H, an unsubstituted aliphatic group, a substituted aliphatic group, an unsubstituted non-aromatic heterocylic group, a substituted non-aromatic heterocylic group, an unsubstituted aryl group, or a substituted aryl group.

26. The method of claim 25, further comprising the steps of:
a) reacting the compound of Formula (I_{INT-A}) with oxalyl chloride to form a product compound represented by the following structural formula: 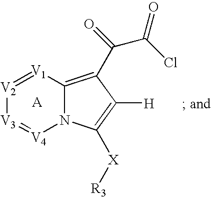
b) amidating the product compound with NHR₁R₂ to form a second product compound represented by the following formula:

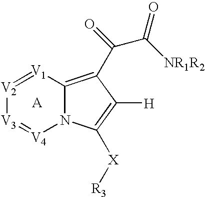

27. The method of claim 25, wherein the compound of Formula (I_{INT-B}) is prepared by reacting a pyridine starting compound and an alkyne starting material in the presence of a catalytic amount of a palladium^{II} salt and a Cu^{I} salt, wherein the starting compound is represented by the following structural formula: 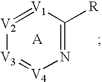
the alkyne starting material is represented by the following structural formula: 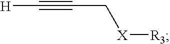
and R is —Br or —I.

28. The method of claim 26, wherein the compound of Formula (I_{INT-B}) is prepared by reacting a pyridine starting compound and an alkyne starting material in the presence of a catalytic amount of a palladium^{II} salt and a Cu^{I} salt, wherein the starting compound is represented by the following structural formula: 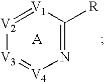
the alkyne starting material is represented by the following structural formula: 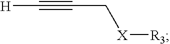
and R is —Br or —I.
